(19) United States
(12) Patent Application Publication
(10) Pub. No.: US 2007/0041937 A1
(43) Pub. Date: Feb. 22, 2007

(54) G-csf derivative for inducing immunological tolerance

(75) Inventors: Geoffrey Hill, Hawthorne (AU); Kellie MacDonald, Wishart (AU); Edward Morris, Long Sutton (GB)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: THE COUNCIL OF THE QUEENSLAND INSTITUTE OF MEDICAL RESEARCH, Herston (AU)

(21) Appl. No.: 10/568,995

(22) PCT Filed: Aug. 20, 2004
(86) PCT No.: PCT/AU04/01116

§ 371 Date: Oct. 12, 2006

(30) Foreign Application Priority Data
Aug. 22, 2003 (AU) 2003904541

(51) Int. Cl. A61K 38/19

(52) U.S. Cl. 424/85.1

(57) ABSTRACT

The invention relates to a method, composition and use thereof for inducing immunological tolerance, in particular transplantation tolerance in a recipient and self-tolerance in a patient. Tolerance is preferably induced by administering a G-CSF derivative, or biologically active fragment, homolog or variant thereof, in particular peg-G-CSF, to a transplantation donor. Transplantation tolerance may reduce or prevent graft versus host disease or graft rejection and self-tolerance may prevent, treat or improve a condition in relation to an autoimmune disorder. The invention also relates to expanding and stimulating selected donor cells by administering a G-CSF derivative, preferably peg-G-CSF. The donor cells are preferably granulocyte-monocyte precursors cells and IL-10 secreting T cells.

### FIELD OF THE INVENTION

THIS INVENTION relates to a method, composition and use thereof for inducing tolerance, including transplantation tolerance in a recipient and self tolerance in a patient. Tolerance is induced by administration of a G-CSF derivative, in particular peg-G-CSF, to a donor or patient. Transplantation tolerance may reduce or prevent graft versus host disease or graft rejection.

### BACKGROUND OF THE INVENTION

Graft versus host disease (GVHD) results in multi-organ damage and immune deficiency significantly impairing overall transplant survival. Allogeneic Stem Cell Transplantation (SCT) is currently indicated in treatment of a number of malignant and non-malignant diseases. However, use of allogenic SCT is limited by serious complications, the most common being GVHD. Use of granulocyte-colony stimulating factor (G-CSF) mobilized stem cell grafts has improved rates of immune and hematopoetic reconstitution, reduced transplant related mortality, and improved leukemia eradication after SCT (Bensinger et al, 2001). The mechanism by which G-CSF reduces GVHD remains controversial. G-CSF has been shown to induce Th2 differentiation in donor T cells prior to SCT and this been suggested to be a protective mechanism from GVHD in this setting (Pan et al, 1995).

Conjugation of a polyethylene glycol (PEG) molecule to a protein (“pegylation”) prolongs the plasma half-life of the conjugated agent (Abuchowski et al, 1977; Bailon et al, 1998), thus reducing frequency of administration of the agent. Peg-G-CSF (also known as peg-filgrastim, peg-Neupogen and Neulasta®, Amgen Inc) has a significantly reduced rate of renal clearance and thus a longer plasma half-life than standard G-CSF (Molineux et al, 2003). Neulasta™ is administered to patients to decrease infection resulting from febrile neutropenia (a decrease in number of white blood cells), in particular in patients with non-myeloid malignancies receiving chemotherapy, in particular, myelosuppressive anti-cancer drugs. U.S. patent application Ser. No. 09/921114 describes treating neutropenia with peg-G-CSF.

A better understanding of the mechanism and cells involved in G-CSF mediated reduction in GVHD is needed to develop new methods and pharmaceutical compositions for treating, preventing or reducing GVHD and autoimmune disorders.

### SUMMARY OF THE INVENTION

It is an object of the invention to provide an alternative or improvement to the abovementioned background art.

The present inventors unexpectedly found that treating a donor with peg-G-CSF is superior to standard G-CSF for inducing tolerance, for example the prevention or reduction of GVHD. Surprisingly, peg-G-CSF enhances biological activity, which is not due to solely due to an increase in half-life. Not being bound by theory, this enhanced biological activity may result from improved binding to the G-CSF receptor and/or activation of selected donor cells. Accordingly, the invention also relates to a surprising discovery that administering a G-CSF derivative, preferably peg-G-CSF, to a donor expands and stimulates selected donor cells including: (1) antigen presenting cells characteristic of granulocyte-monocyte precursors (“GM” cells) as described herein and (2) IL-10 secreting T cells that promote transplantation tolerance and thereby reduce or prevent GVHD in a recipient. The GM cell is preferably characterized by a CD11c negative phenotype, more preferably a CD11b^{pos}/Gr-1^{dim} phenotype. It will also be appreciated that particular aspects of the invention relate to transplantation tolerance and self-tolerance.

In a first aspect, the invention provides a method for inducing transplantation tolerance including the step of administering a G-CSF derivative, or biologically active fragment, homolog or variant thereof, to a donor cell to be transplanted to a recipient.

Preferably, the G-CSF derivative, or biologically active fragment, homolog or variant thereof, comprises recombinant G-CSF.

More preferably, the recombinant G-CSF comprises recombinant human G-CSF.

Even more preferably, the recombinant human G-CSF comprises recombinant methionyl human G-CSF.

In a particular preferred form, the recombinant methionyl human G-CSF is non-glycosylated and is preferably Neupongen®, Amgen Inc.

Preferably, the G-CSF derivative, or biologically active fragment, homolog or variant thereof, comprises peg-G-CSF, or biologically active fragment, homolog or variant thereof.

In a preferred form, the G-CSF derivative, or biologically active fragment, homolog or variant thereof, comprises an N-terminal methionyl residue to which a monomethoxypolyethylene glycol is covalently bound thereto, preferably such form is Neulasta™, Amgen, Inc.

Preferably, the G-CSF derivative comprises G-CSF or a biologically active G-CSF fragment comprising a same amino acid sequence as an amino acid sequence of endogenous G-CSF of the donor.

In a preferred form, the G-CSF derivative or biologically active fragment, homolog or variant thereof, is administered to the donor cell in vivo by administering said G-CSF derivative to a donor.

Preferably, the G-CSF derivative is administered to the donor as a single dose.

Preferably, the G-CSF derivative or biologically active fragment, homolog or variant thereof is administered to the donor in a range from 60 μg/Kg weight of the donor-300 μg/kg weight of the donor, 75 μg/kg -250 μg/kg, 100 μg/kg-225 μg/kg, 125 μg/kg-175 μg/kg or 150 μg/kg-200 μg/kg.

Preferably, the donor is administered the G-CSF derivative or biologically active fragment, homolog or variant thereof between 6 mg-18 mg, 5 mg-20 mg, 8 mg-15 mg or 10 mg-13 mg, wherein said donor is human.

The human donor is preferably administered 6 mg of the G-CSF derivative or biologically active fragment, homolog or variant thereof.

Preferably the human donor weighs more than 45 kg.

Preferably, the G-CSF derivative is peg-G-CSF.

More preferably, the peg-G-CSF is Neulasta™, Amgen Inc.

In one form, the donor cell is isolated from the donor after in vivo administration of the G-CSF derivative or biologically active fragment, homolog or variant thereof.

The donor cell preferably comprises a cell obtained from an organ, blood or tissue, a single cell suspension, unseparated cells, enriched cells and homogeneous cells.

Preferably, the donor cell comprises an immune cell.

In one form, the immune cell is preferably a T cell.

Preferably, administering the G-CSF derivative or biologically active fragment, homolog or variant thereof to the T cell, stimulates the T cell to produce IL-10.

Preferably, the T cell is a regulatory T cell.

Preferably, the regulatory T cell is MHC class II restricted.

In another form, the immune cell is preferably a granulocyte-monocyte.

Preferably, the granulocyte-monocyte is characterized by a CD11c negative phenotype.

More preferably, the granulocyte-monocyte is further characterized by a CD11b^{hi}Gr-1^{dim} phenotype.

Even more preferably, the donor granulocyte-monocyte is still further characterized by a MHC Class I positive, MHC Class II positive, CD80 positive, CD86 positive and CD40 negative phenotype.

The granulocyte-monocyte is preferably capable of stimulating a T cell to produce IL-10.

Preferably, the T cell is a donor T cell.

Preferably, the donor cell comprises non-immune cells that are transplanted before, concurrently and/or after transplanting said immune cells.

Preferably, the non-immune cells comprise a stem cell.

In one form of the invention, the stem cell need not be administered the G-CSF derivative or biologically active fragment, homolog or variant thereof, and said stem cell may be transplanted before, concurrently and/or after transplanting a donor cell having been administered the G-CSF derivative or biologically active fragment, homolog or variant thereof.

The stem cell is preferably obtained from spleen, blood, bone marrow, skin, nasal tissue, hair follicle or other suitable source.

Preferably, the stem cell comprises a hematopoetic stem cell.

In one form of the invention, the donor cell is isolated and purified as an enriched cell population.

Preferably, the enriched cell population comprises a homogeneous cell population.

In another form of the invention, the donor cell is isolated from a donor before administering the G-CSF derivative or biologically active fragment, homolog or variant thereof, to the isolated donor cell.

Another form of the invention further includes the step of propagating the isolated donor cell in vitro before transplantation of the donor cell to the recipient.

Preferably, the donor cell is obtained from a mammal.

Preferably, the recipient is a mammal.

Preferably, the mammal is a human.

The human is preferably a patient.

Induced transplantation tolerance preferably comprises prevention or reduction of graft versus host disease in the recipient.

Preferably, the prevention or reduction of graft versus host disease is greater than that provided by administering G-CSF to the donor.

In a second aspect, the invention provides a method for stimulating a donor T cell to produce IL-10 including the step of administering a G-CSF derivative or biologically active fragment, homolog or variant thereof, to the donor T cell and a donor GM cell to be transplanted to a recipient.

Preferably, the G-CSF derivative or biologically active fragment, homolog or variant thereof comprises recombinant G-CSF.

Preferably, the recombinant G-CSF comprises recombinant human G-CSF.

Preferably, the recombinant human G-CSF comprises recombinant methionyl human G-CSF.

More preferably, the methionyl human G-CSF is not glycosylated and is preferably Neupongen®, Amgen Inc.

Preferably, the G-CSF derivative, or biologically active fragment, homolog or variant thereof, comprises peg-G-CSF, or biologically active fragment, homolog or variant thereof.

In a preferred form, the G-CSF derivative, or biologically active fragment, homolog or variant thereof, comprises an N-terminal methionyl residue to which a monomethoxypolyethylene glycol is covalently bound thereto, preferably such form is Neulasta™, Amgen, Inc.

Preferably, the donor T cell is MHC class II restricted.

Preferably, the donor GM cell is characterized by a CD11c negative phenotype.

More preferably, the GM cell is further characterized by a CD11 b^{hi}Gr-1^{dim} phenotype.

Preferably, the donor cell is obtained from a mammal.

Preferably, the recipient is a mammal.

Preferably, the mammal is a human.

The human is preferably a patient.

The G-CSF derivative or biologically active fragment, homolog or variant thereof, is preferably administered in vivo to a donor before transplantation of the donor T cell to the recipient.

Preferably, the method further includes the step of transplanting said donor T cell and donor GM cell.

Preferably, donor non-immune cells are transplanted to the recipient in addition to the donor T cells and GM cells.

The donor non-immune cells preferably comprise a tissue, organ or cell suspension.

Preferably, the donor non-immune cells are stem cells.

In a third aspect the invention provides a pharmaceutical composition for inducing immunological tolerance when administered to a subject comprising a G-CSF derivative or biologically active fragment, homolog or variant thereof and a pharmaceutically-acceptable carrier.

Preferably, the G-CSF derivative or biologically active fragment, homolog or variant thereof comprises recombinant G-CSF.

Preferably, the recombinant G-CSF comprises recombinant human G-CSF.

More preferably, the recombinant human G-CSF comprises recombinant methionyl human G-CSF.

Even more preferably, the recombinant methionyl human G-CSF is not glycosylated and is preferably Neupongen®, Amgen Inc.

Preferably, the G-CSF derivative comprises peg-G-CSF.

Even more preferably, the G-CSF derivative, or biologically active fragment, homolog or variant thereof, comprises an N-terminal methionyl residue to which a monomethoxypolyethylene glycol is covalently bound thereto, preferably such form is Neulasta™, Amgen, Inc.

Preferably, the subject is human.

Preferably, the subject is a human donor when immunological tolerance comprises transplantation tolerance.

Preferably, the subject is a human patient asymptomatic of an autoimmune disorder when inducing self-tolerance.

Preferably, administering the pharmaceutical composition induces greater immunological tolerance when compared with administering G-CSF.

In a fourth aspect, the invention provides a pharmaceutical composition for inducing immunological tolerance in a subject comprising an isolated cell having been administered a G-CSF derivative or biologically active fragment, homolog or variant thereof.

Preferably, the isolated cell comprises an immune cell.

In one form, the immune cell comprises a T cell.

Preferably, the T cell produces IL-10.

In another form, the immune cell comprises a granulocyte-monocyte.

Preferably, the granulocyte-monocyte is characterized by a CD11c negative phenotype.

More preferably, the granulocyte-monocyte is further characterized by a CD11b^{hi}Gr-1^{dim} phenotype.

In one form, immunological tolerance comprises transplantation tolerance.

Preferably, transplantation tolerance prevents or reduces graft versus host disease.

Preferably, the isolated cell is obtained from a donor.

More preferably, the donor is human.

Preferably, the subject is a recipient.

More preferably, the recipient is human.

In another form, immunological tolerance comprises self-tolerance.

Preferably self-tolerance reduces or prevents symptoms of an autoimmune disorder.

Preferably, the autoimmune disorder is selected from the group consisting of rheumatoid arthritis, systemic lupus erythematosus, multiple sclerosis and inflammatory bowel disease.

Preferably, the isolated cell is obtained from the subject.

Preferably, the subject is a patient.

More preferably, the patient is a human patient.

Even more preferably, the human patient is asymptomatic for an autoimmune disorder.

In a fifth aspect, the invention provides use of the pharmaceutical composition of the third aspect to induce immunological tolerance.

In a sixth aspect, the invention provides use of the pharmaceutical composition of the fourth aspect to induce immunological tolerance.

In a seventh aspect, the invention provides a method of transplantation including the steps of:

(1) administering to a donor a pharmaceutical composition comprising a G-CSF derivative or biologically active fragment, homolog or variant thereof and a pharmaceutically-acceptable carrier;

(2) isolating a cell, tissue or organ from said donor; and

(3) transplanting said cell, tissue or organ to a recipient.

Preferably, the G-CSF derivative or biologically active fragment, homolog or variant thereof comprises recombinant G-CSF derivative or biologically active fragment, homolog or variant thereof.

More preferably, the recombinant G-CSF derivative or biologically active fragment, homolog or variant thereof comprises human G-CSF.

Even more preferably, the G-CSF derivative or biologically active fragment, homolog or variant thereof comprises peg-G-CSF derivative or biologically active fragment, homolog or variant thereof.

Preferably, the donor and recipient are human.

The cells are preferably isolated from the donor and propagated in culture before transplanting said cells to the recipient.

In one form of the invention, transplantation comprises heterologous transplantation wherein the donor and recipient are different individuals.

In another form of the invention, transplantation comprises autologous transplantation wherein the donor and recipient are the same individual.

In an eighth aspect, the invention provides a method for inducing self-tolerance in a patient including the step of administering a G-CSF derivative or biologically active fragment, homolog or variant thereof, to the patient.

Preferably, inducing self-tolerance in the patient prevents, treats or reduces an autoimmune disorder of the patient.

Preferably, the patient is asymptomatic of an autoimmune disorder.

Preferably, the autoimmune disorder is selected from the group consisting of rheumatoid arthritis, systemic lupus erythematosus, multiple sclerosis and inflammatory bowel disease.

Preferably, the G-CSF derivative or biologically active fragment, homolog or variant thereof stimulates an immune cell of the patient to thereby induce self-tolerance.

In one form, the immune cell comprises a T cell.

More preferably, the T cell is stimulated to produce IL-10.

In another form, the immune cell comprises a granulocyte-monocyte cell.

Preferably, the granulocyte-monocyte is characterized by a CD11c negative and CD11b^{hi}Gr-1^{dim} phenotype

Preferably, the immune cell of the patient is isolated from the patient, propagated in vitro and administered to the patient.

Preferably, the G-CSF derivative or biologically active fragment, homolog or variant thereof comprises peg-G-CSF or biologically active fragment, homolog or variant thereof.

More preferably, the peg-G-CSF comprises peg-human G-CSF or biologically active fragment, homolog or variant thereof.

Even more preferably, the peg-human G-CSF or biologically active fragment, homolog or variant thereof comprises peg-recombinant human G-CSF or biologically active fragment, homolog or variant thereof.

Still more preferably, the peg-recombinant human G-CSF comprises neulasta™.

Throughout this specification unless the context requires otherwise, the word “comprise”, and variations such as “comprises” or “comprising”, will be understood to imply the inclusion of the stated integers or group of integers or steps but not the exclusion of any other integer or group of integers.

### BRIEF DESCRIPTION OF THE FIGURES

In order that the invention may be readily understood and put into practical effect, preferred embodiments will now be described by way of example with reference to the accompanying figures.

FIG. 1A: Survival by Kaplan-Meier analysis. Donor B6 mice were treated for 6 days with human G-CSF (0.2 ug/animal, 2 μg/animal or 10 μg/animal) or control diluent. T cell dose was equilibrated across all groups (3×10⁶ T cells/recipient). Splenocytes were harvested on day 7 and transplanted into lethally irradiated (1100 cGy) B6D2F1 recipient mice (control syngeneic recipients n=6; control allogeneic n=6; G-CSF 0.2 μg/day n=12; G-CSF 2.0 μg/day n=12; G-CSF 10 μg/day n=6). P=0.03, 0.2 μg G-CSF versus 2 μg G-CSF; P=0.004, 0.2 μg G-CSF versus 10 μg G-CSF. Combined results from two identical experiments shown.

FIG. 1B: Survival by Kaplan-Meier analysis. Donor B6 mice were treated with murine G-CSF (0.2 μg/animal, 0.5 μg/animal or 2 μg/animal for 6 days) or control diluent and transplanted as above. B6D2F1 recipient mice (control syngeneic recipients n=6; control allogeneic n=6; murine G-CSF 0.2 μg/day n=6; murine G-CSF 0.5 μg/day n=6; murine G-CSF 2 μg/day n=12). Survival P=0.003, 0.2 μg murine G-CSF versus 2 μg murine G-CSF. Combined results from 2 identical experiments shown.

FIG. 2A: Survival by Kaplan-Meier analysis. Donor B6 mice received either control diluent, 2 μg standard human G-CSF daily for 6 days, 3 μg peg-G-CSF or 12 μg peg-G-CSF as a single injection on day —6. Lethally irradiated B6D2F1 recipient mice were transplanted as in FIG. I (control syngeneic recipients n=6; control allogeneic n=6; peg-G-CSF 3 μg n=6; peg-G-CSF 12 μg n=6; human standard G-CSF 2 μg/day n=18. P=0.82, 3 μg peg-G-CSF versus 12 μg peg-G-CSF, P=0.0001, 2 μg G-CSF (for 6 days) versus 12 μg peg-G-CSF (single dose).

FIG. 2B: GVHD clinical scores were determined as a measure of GVHD severity in surviving animals as described herein. *P<0.05 for 2 μg human G-CSF (6 days) versus 12 μg peg-G-CSF (single dose). Combined results from 2 identical experiments shown.

FIG. 3A: Splenocyte expansion following donor pre-treatment with standard or pegylated G-CSF shown as relative proportions of each cell lineage. Donor B6 mice (n=4 per group) received either control diluent, 2 μg human G-CSF/day for 6 days or single injection of 12 μg peg-G-CSF day —6 and splenocytes were harvested on day 7.

FIG. 3B: Splenocyte expansion following donor pre-treatment with standard or pegylated G-CSF shown as absolute numbers of each cell lineage. *P<0.05 control versus peg-G-CSF, +P<0.05 peg-G-CSF versus control and G-CSF. Data presented as mean ±SD.

FIG. 4 shows donor GM cells from donors administered peg-G-CSF (12 μg once only on day 6) prior to transplantation prevent GVHD. 10⁶ donor GM cells from peg-G-CSF treated mice were sort purified by FACS (resulting GM cells were characterized by a CD11c negative and CD11b^{hi}Gr-1^{dim} phenotype) and added to splenocytes from control treated allogeneic B6 animals (n=5). Cohorts of GVHD controls received unseparated splenocytes from control treated allogeneic donors without GM (control allo, n=5). **P<0.01.

FIG. 5A: Donor treatment with peg-G-CSF impairs T cell function and induces regulatory T cell activity. C57BL/6 T cells from control, G-CSF 2 μg/day for 6 days or peg-G-CSF 12 μg single dose day —6 were stimulated at ratios as shown with irradiated B6D2F1 peritoneal macrophages. Proliferation was measured at 72 hours via standard [³H] Thymidine incorporation assay. P<0.05 control versus G-CSF and P<0.05 control versus peg-G-CSF.

FIG. 5B shows IFN-γ production determined in culture supernatants from experiments in relation to FIG. 5A by ELISA.

FIG. 5C shows IL-2 production determined in culture supernatants from experiments in relation to FIG. 5A by ELISA.

FIG. 5D: Non-cytokine exposed C57BL/6 T cells were stimulated with irradiated B6D2F1 macrophages. Additional T cells from wild-type C57BL/6 donors pre-treated with control diluent or peg-G-CSF 12 μg day —6, or from IL-10^{-/-} donors pre-treated with peg-G-CSF 12 μg day —6, were added at doses as shown. Proliferation was measured at 72 hours via standard [³H] Thymidine incorporation assay. *P<0.05 control versus wild-type peg-G-CSF.

FIG. 5E: Whole spleen from control, G-CSF, or peg-G-CSF pre-treated donors as above was stimulated with LPS and CPG, and IL-10 measured in supernatants at 48 hours by ELISA. P=0.0002 control versus G-CSF; P=0.001 control versus peg-G-CSF. Data (FIGS. 11A-11C) presented as mean ±SD of triplicate wells and represents one of two identical experiments.

FIG. 6A: Survival by Kaplan-Meier analysis. P<0.001 for wild-type TCD spleen+wild-type T cells versus wild-type TCD spleen+IL10^{-/-} T cells; P<0.0001 IL10^{-/-} TCD spleen+wild-type T cells versus IL10^{-/-} spleen+IL10^{-/-} T cells. FIG. 6A shows protection from GVHD afforded by peg-G-CSF is dependant on donor T cell production of IL-10. Donors were pre-treated with a single dose of 12 g peg-G-CSF at day —6. T cell depleted (TCD) splenocytes from wild-type or IL-10^{-/-} donors plus purified CD3^{pos} T cells from wild-type or IL-10^{-/-} B6 donors were combined as indicated, and injected into lethally irradiated B6D2F1 recipients (wild-type TCD spleen only n=6, wild-type T cells plus wild-type or IL-10^{-/-} spleen n=15, IL-10^{-/-} T cells plus wild-type or IL-10^{-/-} spleen n=13).

FIG. 6B: GVHD clinical scores determined as a measure of GVHD severity in surviving animals. *P<0.05 wild-type TCD spleen+IL10^{-/-} T cells versus wild-type TCD spleen+wild-type T cells.

FIG. 7: The protective IL-10 producing donor T cell has regulatory function. Lethally irradiated B6D2F1 recipients received splenocytes from control treated wild-type B6 donors plus additional purified T cells from control or cytokine pre-treated donors, as shown (syngeneic control n=3; allogeneic control n=5; wild-type allogeneic control+wild-type control pre-treated T cells n=9; wild-type allogeneic control+wild-type G-CSF pre-treated T cells n=10; wild-type allogeneic control+wild-type peg-G-CSF pre-treated T cells n=14; wild-type allogeneic control+IL-10^{-/-} peg-G-CSF pre-treated T cells n=13). Survival by Kaplan-Meier analysis. P<0.0001 wild-type allogeneic control+wild-type peg-G-CSF pre-treated T cells versus wild-type allogeneic control+wild-type control pre-treated T cells; P<0.0001 wild-type allogeneic control+wild-type peg-G-CSF pre-treated T cells versus wild-type allogeneic control+wild-type G-CSF pre-treated T cells; P<0.0001 wild-type allogeneic control+wild-type peg-G-CSF pre-treated T cells versus wild-type allogeneic control+IL-10^{-/-} peg-G-CSF pre-treated T cells. Data combined from 2 identical experiments.

FIG. 8A shows mobilisation in human patients of CD34⁺ stem cells into the blood 3 to 6 days after administration of peg-G-CSF and confirmed that CD34⁺ counts peaked 5 days after peg-G-CSF administration. The dotted lines represent a range of CD34⁺ stem cell mobilisation the study aimed at achieving.

FIG. 8B shows total number of CD34⁺ stem cells harvested from human donors by standard aphaeresis of 1.5 blood volumes on days 5 and 6 and pooled prior to transplantation. The total CD34⁺ collections for individual days and combined are shown. The dotted lines represent the range of total CD34⁺ numbers the study aimed at collecting (4-8×10⁶/kg recipient body weight).

FIG. 8C shows human donor haematopoietic engraftment as days after transplantation by neutrophil and platelet recovery (>0.5×10⁶/l and >20×10⁹/l respectively) and was within the range expected from historical controls (shown by dotted lines).

### DETAILED DESCRIPTION OF THE INVENTION

Unless defined otherwise, all technical and scientific terms used herein have a meaning as commonly understood by those of ordinary skill in the art to which the invention belongs. Although any method and material similar or equivalent to those described herein can be used in the practice or testing of the present invention, preferred methods and materials are described. For the purpose of the present invention, the following terms are defined below.

The present invention relates to an unexpected finding that treating a donor with peg-G-CSF is markedly superior to G-CSF for the induction of transplantation tolerance and prevention of GVHD. Accordingly, peg-G-CSF does not merely increase G-CSF half-life, but surprisingly also enhances G-CSF biological activity, possibly by different processing or internalization of the peg-G-CSF. It will be appreciated that PEG preferably is conjugated to any suitable biologically active form of G-CSF, including for example, a G-CSF fragment, homolog or variant thereof. Such a biologically active form of G-CSF preferably is capable of binding the G-CSF receptor. The invention also relates to a discovery that tolerance may be induced by stimulating and expanding a sub-set of donor antigen presenting cells, namely GM cells as described herein, by administering G-CSF, preferably a G-CSF derivative such as peg-G-CSF, to a patient. Not being bound by theory, the GM cells are hypothesized to promote transplantation tolerance by induction of MHC class II restricted IL-10 producing T cells.

G-CSF and G-CSF Derivatives

By “protein” is also meant “polypeptide”, either term referring to an amino acid polymer, comprising natural and/or non-natural D- or L-amino acids as are well understood in the art. G-CSF may be referred to as both a protein or polypeptide. Protein may refer to a peptide or fragments thereof, for example a fragment of G-CSF.

“G-CSF” refers to G-CSF protein and fragments, homologs and variants thereof. G-CSF protein is distinct from a G-CSF derivative, for example peg-G-CSF. G-CSF is not artificially conjugated to another molecule, for example PEG as described herein. G-CSF protein may comprise naturally occurring modification such as glycosylation, but in a preferred form is nonglycosylated and expressed from a bacteria cell. G-CSF may be derived from any species, including human, mouse, rat and others. A preferred form of G-CSF is human G-CSF for use in humans. G-CSF may be recombinant or native and may comprise natural and/or non-natural D- or L-amino acids as are well understood in the art. Recombinant human G-CSF comprises recombinant methionyl human G-CSF (Neupogen®, Amgen Inc). NEUPOGEN® is an Amgen Inc. trademark for recombinant methionyl human granulocyte colony-stimulating factor (r-metHuG-CSF). NEUPOGEN® comprise a recombinant 175 amino acid protein having a molecular weight of 18,800 daltons, which is non-glycosylated and produced by E. col. The protein has an amino acid sequence identical to a predicted human amino acid sequence for G-CSF, with an additional N-terminal methionine that is required expression in E coli. NEUPOGEN® is non-glycosylated, which is different than endogenous human G-CSF. G-CSF suitable for use with the present invention comprises all known forms of the protein from any species, preferably; mouse and human G-CSF. Examples of preferably forms of G-CSF include NEUPOGEN®, G-CSF referred to in Bensinger et al, 2001 and G-CSF having accession number Q99062, NCBI, which are incorporated herein by reference.

The protein may be isolated, for example, G-CSF or G-CSF derivative and other proteins may be removed from their natural state or be synthetically made or recombinantly expressed. A “peptide” is a protein having no more than fifty (50) amino acids.

In one embodiment, a “fragment” includes an amino acid sequence that constitutes less than 100% of a polypeptide, including forms comprising less than or equal to 99%, 98%, 97%, 96%, 95%, 90%, 85%, 80%, 75%, 70%, 65%, 60%, 55%, 50%, 45%, 40%, 35%, 30%, 25%, 20%, 15%, 10% or 5% of a total length of a protein. A fragment encompasses a sub-fragment. In a preferred form, the fragment comprises at least 20%, preferably at least 30%, more preferably at least 80% or even more preferably at least 90%, 95%, 96%, 97%, 98% or even 99% of said polypeptide. A preferred fragment comprises a biologically active domain of G-CSF, including for example a G-CSF receptor binding domain and essential amino acids thereof, and domains required for G-CSF receptor activation.

The fragment includes a “biologically active” fragment or fragment which retains “biological activity” of a given protein or peptide. For example, a biologically active fragment of G-CSF capable of inducing immunological tolerance in a subject may be used in accordance with the invention. Biological activity of a biologically active fragment of G-CSF may be assessed by binding to a G-CSF receptor or fragment thereof, being capable of being bound by an anti-G-CSF antibody, administration to a donor before transplantation and assessing GVHD, assessing a downstream event following binding to a G-CSF receptor or any other suitable assay known in the art capable of assessing a biological response by the fragment. An example of a biologically active G-CSF fragment includes a domain capable of binding the G-CSF receptor. A biologically active form of G-CSF need not satisfy all of the abovementioned methods of assessment to be considered biologically active and the above assessment method are merely examples. Binding to a G-CSF receptor or binding by an anti-G-CSF antibody may be assessed by known methods including ELISA, dot blots and the like. Preferably, the biologically active fragment, homolog or variant of G-CSF is capable of binding to a G-CSF receptor of the donor, recipient or patient. The biologically active fragment constitutes at least greater than 1% of the biological activity of the entire polypeptide or peptide, preferably at least greater than 10% biological activity, more preferably at least greater than 25% biological activity and even more preferably at least greater than 50%, 60%, 70%, 75%, 80%, 85%, 90%, 95%, 96%, 97%, 98%, 99% or even 100% biological activity. A biologically active fragment in one form may have a biological activity greater than that of a full-length protein.

The term “biologically active” and “biological activity” may also be used when referring to a derivative, homolog and variant of a protein, the term meaning the same as set out above in relation to a protein fragment and the same assays may be used to assess biological activity. Again, the assays described herein are merely examples of some assays that may be used to assess biological activity and to be biologically active, a derivative, homolog or variant need to satisfy all of the assessment methods. A biologically active derivative, homolog or variant preferably retains at least greater than 1% of the biological activity of a reference protein, such as human G-CSF. A homolog or an ortholog of human G-CSF, for example mouse G-CSF, when administered to a human may have a biological activity similar to human G-CSF or a biological activity less than that of human G-CSF. It will also be appreciated that “biological activity” encompasses an enhanced biological activity, for example, a G-CSF derivative such as peg-G-CSF preferably has an enhanced biological activity when compared with G-CSF, preferably enhanced immunological tolerance. Preferably, the enhanced immunological tolerance comprises transplantation tolerance and self-tolerance. Accordingly, the term biological activity encompasses greater than 100% biological activity when compared with a reference protein, for example G-CSF.

As used herein, “variant” proteins are proteins in which one or more amino acids have been replaced by different amino acids. Protein variants of G-CSF that retain biological activity of native or wild type G-CSF may be used in accordance with the invention. It is well understood in the art that some amino acids may be changed to others with broadly similar properties without changing the nature of the activity of the polypeptide (conservative substitutions). Generally, the substitutions which are likely to produce the greatest changes in a polypeptide's properties are those in which (a) a hydrophilic residue (e.g., Ser or Thr) is substituted for, or by, a hydrophobic residue (e.g. Leu, Ile, Phe or Val); (b) a cysteine or proline is substituted for, or by, any other residue; (c) a residue having an electropositive side chain (e.g., Arg, His or Lys) is substituted for, or by, an electronegative residue (e.g., Glu or Asp) or (d) a residue having a bulky side chain (e.g., Phe or Trp) is substituted for, or by, one having a smaller side chain (e.g., Ala, Ser)or no side chain (e.g., Gly).

Polypeptide and Nucleic Acid Sequence Comparison

Terms used herein to describe sequence relationships between respective nucleic acids and polypeptides include “comparison window”, “sequence identity”, “percentage of sequence identity” and “substantial identity”. Because respective nucleic acids/polypeptides may each comprise (1) only one or more portions of a complete nucleic acid/polypeptide sequence that are shared by the nucleic acids/polypeptides, and (2) one or more portions which are divergent between the nucleic acids/polypeptides, sequence comparisons are typically performed by comparing sequences over a “comparison window” to identify and compare local regions of sequence similarity. A “comparison window” refers to a conceptual segment of typically at least 6 contiguous residues that is compared to a reference sequence. The comparison window may comprise additions or deletions (i.e., gaps) of about 20% or less as compared to the reference sequence (which does not comprise additions or deletions) for optimal alignment of the respective sequences. Optimal alignment of sequences for aligning a comparison window may be conducted by computerised implementations of algorithms (for example ECLUSTALW and BESTFIT provided by WebAngis GCG, 2D Angis, GCG and GeneDoc programs, incorporated herein by reference) or by inspection and the best alignment (i.e., resulting in the highest percentage homology over the comparison window) generated by any of the various methods selected.

The ECLUSTALW program is used to align multiple sequences. This program calculates a multiple alignment of nucleotide or amino acid sequences according to a method by Thompson, J. D., Higgins, D. G. and Gibson, T. J. (1994). This is part of the original ClustalW distribution, modified for inclusion in EGCG. The BESTFIT program aligns forward and reverse sequences and sequence repeats. This program makes an optimal alignment of a best segment of similarity between two sequences. Optimal alignments are determined by inserting gaps to maximize the number of matches using the local homology algorithm of Smith and Waterman. ECLUSTALW and BESTFIT alignment packages are offered in WebANGIS GCG (The Australian Genomic Information Centre, Building JO3, The University of Sydney, N.S.W 2006, Australia).

Reference also may be made to the BLAST family of programs as for example disclosed by Altschul et al., 1997, Nucl. Acids Res. 25 3389, which is incorporated herein by reference. A detailed discussion of sequence analysis can be found in Chapter 19.3 of Ausubel et al, supra.

The term “sequence identity” is used herein in its broadest sense to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G, U) occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for windows; available from Hitachi Software engineering Co., Ltd., South San Francisco, Calif., USA). It will be appreciated that determining sequence similarity or sequence identity may be useful in assessing and selecting candidate homologs that may be useful in relation to the present invention.

As generally used herein, a “homolog” shares a definable nucleotide or amino acid sequence relationship with another nucleic acid or polypeptide as the case may be. A “protein homolog” preferably shares at least 70% or 80% sequence identity, more preferably at least 85%, 90% and even more preferably at least 95%, 96%, 97%, 98% or 99% sequence identity with the amino acid sequences of polypeptides as described herein. Homologs of G-CSF may also be used in accordance with the invention. Such G-CSF homologs would preferably be characterized by biological activity about the same or greater than that of a G-CSF protein having a high or substantial biological activity.

“Orthologs” are included within the scope of homologs. Orthologs are functionally-related proteins and their encoding nucleic acids, isolated from other organisms or species. For example, human G-CSF is an ortholog of mouse G-CSF. It will be appreciated that a protein ortholog may be administered to a donor and retain biological activity. However, it is preferred that the G-CSF administered comprises an amino acid sequence that is the same or similar to that of the donor, and preferably the same or similar to that of a recipient. More preferably, the G-CSF is human G-CSF and the human G-CSF is administered to a human donor, human recipient or human patient. An example of a suitable human G-CSF includes Neupogen®), Amgen Inc, and human G-CSF described in U.S. patent application Ser. No. 09/921,114, incorporated herein by reference.

With regard to protein variants, these can be created by mutagenising a polypeptide or by mutagenising an encoding nucleic acid, such as by random mutagenesis or site-directed mutagenesis. Examples of nucleic acid mutagenesis methods are provided in Chapter 9 of CURRENT PROTOCOLS IN MOLECULAR BIOLOGY, Ausubel et al., supra which is incorporated herein by reference.

As used herein, “derivative” proteins are proteins that have been altered, for example by conjugation or complexing with moieties or by post-translational modification techniques as would be understood in the art. Moieties include synthetic and natural polymers such as polyethylene glycol (PEG), polyvinyl pyridine (PVP), poly[N-(2-hydroxypropyl)methacrylamide, microspheres, liposomes, nanoparticles, dextrans and fusion proteins. A preferred derivative includes G-CSF conjugated to polyethylene glycol (PEG) (i.e. “pegylation”), resulting in peg-G-CSF as described herein. Embodiments of the invention include one or more linear and branching forms of PEG conjugated to G-CSF or biologically active fragment, homolg or variant thereof. Accordingly, one or more moieties of PEG may be attached to a protein, for example embodiments of the invention included G-CSF or biologically active fragment, homolog or variant thereof conjugated to one, two, three, four, five, six, seven, eight, nine, ten or more PEG moieties (linear and/or branching) of a same or different molecular weight. A preferred peg-G-CSF is Neulasta™, available from Amgen Inc, incorporated herein by reference. Neulasta™ (also referred to as peg-filgrastim) is a covalent conjugate of recombinant methionyl human G-CSF (Neupogen®) and monomethoxypolyethylene glycol. To make Neulasta™ (peg-G-CSF), a 20 kd monomethoxypolyethylene glycol molecule is covalently bound to an N-terminal methionyl residue of human G-CSF (Neupogen®). An average molecular weight of peg-filgrastim (Neulasta™) is approximately 39 kd. It will be appreciated that PEG may be conjugated to any suitable agent capable of binding a G-CSF receptor, for example forms of G-CSF, including a G-CSF fragment, preferably a biologically active fragment, homolog, ortholog, variant and G-CSF mimetic. Preferably, binding to the G-CSF receptor results in biologically activating a cell expressing the G-CSF receptor. A preferred form of peg-G-CSF and methods for making peg-G-CSF are described in U.S. patent application Ser. No. 09/921,114, incorporated herein by reference. As described in this US Patent Application, PEG may be covalently bound to amino acid residues of G-CSF, preferably human G-CSF. The amino acid residue may be any reactive one having, for example, free amino or carboxyl groups, to which a terminal reactive group of an activated polyethylene glycol may be bound. The amino acid residues having the free amino groups may include lysine residues and N-terminal amino acid residue, and those having the free carboxyl group may include aspartic acid, glutamic acid residues and C-terminal amino acid residue. A molecular weight of PEG is not limited to any particular value or range; however, a suitable range includes from 0.5-170 kd, 1-100 kd, 5-80 kd, 10-60 kd, 20-50 kd and 30-40 kd. The molecular weight of PEG may be any value between the indicated ranges, for example, 1 kd, 5 kd, 6 kd, 10 kd, 15 kd, 20 kd, 50 kd, 100 kd or 170 kd. Preferred molecular weights include 6 kd, 20 kd, 50 kd and 170 kd, most preferably 20 kd as used for Neulasta™. It will be appreciated that selecting PEG of different molecular weights may affect half life of the attached protein and may affect biological activity thereof. For example, binding to the G-CSF receptor and/or internalization of the bound G-CSF by a cell may vary depending on the molecular weight of the PEG. Accordingly, a person skilled in the art may select a suitable molecular weight of PEG, for example 20 kd as discussed above. Forms of peg-G-CSF that may preferably be used with the invention include Neulasta™ and forms described in Abuchowski et al, 1977; Bailon et al, 1998 and Molineux et al, 2003.

PEG may be bound to G-CSF via a terminal reactive group, linker or a spacer as is known in the art. The spacer may mediate a bond between the free amino or carboxyl groups and polyethylene glycol. Peg-G-CSF may be purified from a reaction mixture using methods common in the art for purifying proteins, such as affinity purification, dialysis, salting-out, ultrafiltration, ion-exchange chromatography, gel chromatography and electrophoresis. Ion-exchange chromatography is particularly effective in removing unreacted polyethylene glycol and human G-CSF. Peg-G-CSF is also commercially available, for example from Amgen Inc, Thousand Oaks, Calif., USA and sold under the trade mark name of Neulasta™, as described herein.

Derivatives also comprise amino acid deletions and/or additions to polypeptides of the invention, or variants thereof. “Additions” of amino acids may include fusion of the protein with amino (N) and/or carboxyl (C) terminal amino acids “tags” or proteins. An example of a G-CSF derivative comprising a fusion protein comprises albumin-GCSF (Albugranin™, Human Genome Sciences).

Other derivatives contemplated by the invention include, modification to amino acid side chains, incorporation of unnatural amino acids and/or their derivatives during peptide or polypeptide synthesis and the use of cross linkers and other methods which impose conformational constraints on the polypeptides, fragments and variants of the invention.

An “agonist” refers to a molecule, such as a drug, enzyme activator or protein, which enhances activity of another molecule or receptor site. For example, G-CSF and peg-G-CSF are agonists of the G-CSF receptor.

Cells Used in Relation to the Invention

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state.

Cells used in relation to the invention may be isolated from a donor before, concurrently and/or after treatment with the G-CSF derivative. The isolated cells may be isolated from blood using well know methods in the art. The isolated cells may form part of a tissue or organ, for example a biopsy from bone, spleen or any other tissue. Accordingly, isolated cells may comprise an isolated heterogeneous population of cells, an isolated homogeneous population of cells, cell suspension, unseparated cells and other forms of isolated cells well known in the art. It will be appreciated that use of the term “cell” includes one or more cells, for example a single cell, a population of cells and a group of cells that may form a tissue or organ. In a preferred embodiment, isolated cells comprise isolated immune cells, in particular isolated T cells and GM cells from a donor. The isolated T cells and GM cells may be isolated as purified or homogenous cell populations or may be isolated as part of a tissue or organ, for example may form part of a transplanted tissue or organ from a donor to a recipient.

Isolated material includes cells that have been “enriched” or “purified”, meaning a population of cells comprising a higher percentage of a particular cell type when compared with other individual cell types from a same tissue or origin. An enriched or purified population of cells preferably comprises at least 5%, more preferably at least 10%, 15%, 20%, 25%, 50% or greater of a particular cell type when compared with other cell types of a total population.

An enriched or purified cell population may be homogeneous for a selected cell type. A “homogeneous” cell population may in one embodiment be referred to as a “substantially homogeneous” cell population, which preferably comprises a single cell type comprising at least 25% of the total isolated cell population, more preferably at least 50%, even more preferably at least 75%, at least 80%, at least 90%, 91%, 92%, 93%, 94%, 95%, 96%, 97%, 98% or 99% and most preferably 100% of the total isolated cell population. Although an enriched population and homogeneous population of cells may refer to a same percentage of a same cell type in a total population, the term homogeneous is used herein to generally refer to a cell population comprising a greater percentage or number of same cells in a total cell population, preferably greater than 90%, inclusive of all values between 90% and 100%.

It will be appreciated that a total isolated cell population may comprise multiple cell types, accordingly, an enriched or homogeneous cell population comprising less than 50% of the total isolated cell population may nevertheless comprise a greater percentage or greater number of a same cell type when compared with other cell types in the total isolated cell population. T cells, granulocyte-monocyte, macrophages and/or stem cells may be enriched from spleen, bone or any other suitable tissue or organ and in one embodiment preferably comprise a homogeneous cell population.

Cells may be purified using any suitable method known in the art, including for example, affinity purification using a ligand, protein, antibody (either monoclonal or polyclonal) or any other suitable binding agent capable of binding to a selected cell. The binding agent may be attached to a substrate such as a matrix, bead (including a magnetic bead), solid surface or any other suitable surface. The cells may be purified using an affinity column, panning, FACS and like methods known in the art. Cells may be purified by cell deletion by binding unwanted bound cells to a binding agent and discarding the bound cells. Alternatively, or in addition, cells may be purified by positively selecting cells by binding wanted cells to a binding agent and collecting the bound cells. The bound cells may further be removed from the binding agent. Cells may be purified by separation based on size, density or other physical property, for example by density gradient, including nycodenz density gradient. Cells, for example T cells, may be purified by teased nylon wool column purification.

“Stem cell” as used herein refers to a “multipotent” cell capable of giving rise to many different types of cells. A stem cell may be obtainable from any suitable source, including for example, spleen, blood, bone marrow, skin, nasal tissue, hair follicle and any other source. A stem cell may be an allogeneic stem cell. Stem cells may be used in allogeneic stem cell transplantation (SCT) as is known in the art and described herein. The G-CSF derivate treated donor T cells and/or granulocyte-monocyte may be transplanted with allogeneic stem cells to reduce or prevent GVHD. The stem cell is preferably a hematopoietic stem cell.

By “antigen presenting cell” (APC) is meant a cell that displays a foreign antigen on its cell surface, typically bound to a class II glycoprotein. The foreign antigen may be recognized by a helper T cell. A granulocyte-monocyte and dendritic cell are APC.

By “T cell” is also meant “T lymphocyte”, which refers to a thymus-derived lymphocyte involved with cell-mediated immune responses. T cell includes: cytotoxic T cells, regulatory T cells, helper T cells and suppressor T cells. A granulocyte-monocyte cell of the invention preferably is capable of stimulating a MHC class II T cell to secrete IL-10. A preferred T cell of the invention is a regulatory T cell.

By “granulocyte-monocyte” (“GM”) is meant a type of white blood cell, namely a precursor cell in the developmental pathway of becoming a monocyte. Preferably, a GM cell as used herein is characterized by a CD11c negative phenotype. More preferably, the GM cell is further characterized by a CD11b^{hi}Gr-1^{dim} phenotype. In one embodiment, the GM cells is characterized by a MHC Class I positive, MHC class II positive, CD80 positive, CD86 positive and CD40 negative phenotype. Preferably, the GM cell is a donor cell as described herein. Preferably, the GM cell is obtained from a human donor also referred to as a human patient. In one embodiment of the invention, the GM cell is isolated from a human donor before transplantation to a human recipient.

By “dendritic cell” (DC) is meant a type of APC that have a function in the development of immune responses against microbial pathogens and tumors. Subpopulations of DC may be present in the thymus, spleen, Peyer's patches, lymph nodes and skin. A DC cell preferably positively expresses CD11c.

Cells used in relation to the invention, either treated and/or untreated, may be propagated in vitro before transplantation. Cells may be propagated using tissue culture methods that are well known in the art. Cells may be propagated on any suitable surface, including tissue culture in flasks, plates, wells, roller bottles and other known means in the art. The surface may be uncoated, glass, polymer or coated with a suitable molecule such as a matrix (eg extracellular matrix), charged particle (eg poly-l-lysine) and the like that may be selected by a skilled person. The cells are preferably propagated in culture media comprising actives including antibiotics, growth factors, cytokines and other actives that may increase the rate of cell division, differentiate the cells into a selected cell type and/or maintain a cell as a selected cell type. Preferably, the donor cells of the invention are cultured in media comprising GM-CSD, IL-10 and/or TGF-β.

Pharmaceutical Compositions in Relation to the Invention

A “composition” includes a “pharmaceutical composition”, which comprises an active for delivery to a subject. The active may be a protein such as G-CSF, or a biologically active fragment, homolog, variant or derivative thereof, such as peg-G-CSF, which stimulates a biological activity. A preferred form of a pharmaceutical composition comprises a G-CSF derivative, more preferably peg-G-CSF, including Neulasta™, Amgen Inc. However, the composition may comprise other forms of G-CSF, including for example G-CSF conjugated to other polymers and moieties such as albumin and biologically active fragments, variants and homologs thereof. The composition may further comprise non-PEG forms of G-CSF and G-CSF mimetics, and respective fragments, variants and homologs thereof in addition to the G-CSF derivative. The pharmaceutical composition may also comprise other actives commonly used in transplantation, in particular transplantation of stem cells.

The composition may also comprise as an active one or more cells, for example one or more cells such as donor T cells and/or donor granulocyte-monocytes that have been administered with a G-CSF derivative (eg peg-G-CSF). Preferably, the T cells are regulatory T cells as described herein, preferably secreting IL-10. Preferably the GM cells are characterised by a CD11c negative phenotype, more preferably the GM cell is further characterized by a CD11b^{hi}Gr-1^{dim} phenotype. It will be appreciated that in addition to treatment with the G-CSF derivative, the cells of the composition may further be treated with G-CSF and/or a G-CSF mimetic, or other active. The composition may comprise a homogeneous population of cells treated in accordance with the invention. For example, the composition in one embodiment comprises a homogeneous population of T cells and/or a homogeneous population of GM cells isolated from a donor treated with a G-CSF derivative in accordance with the invention. The composition in another form comprises a heterogeneous population of cells. The heterogeneous population of cells in one form are non-purified cells, including a tissue and organ. The heterogeneous population of cells in another form comprises two or more homogenous population of cells that have been combined to thereby form the heterogeneous population. Such a combined heterogeneous population may comprise, two, three, four, five, six, seven, eight, nine, ten, or more homogeneous populations. For example, a homogenous population of treated donor T cells, GM cells and/or a homogeneous population of allogeneic stem cells. The composition in one embodiment comprises a heterogeneous population of cells wherein some of the cells have been treated in accordance with the invention, for example T cells and/or GM cells, and untreated cells, for example allogeneic stem cells. A heterogeneous population of cells in one embodiment comprises one or more heterogeneous populations of cells and one or more homogeneous population of cells. For example, one, two, three, four, five, six, seven, eight, nine, ten or more heterogeneous populations of cells and one, two, three, four, five, six, seven, eight, nine, ten or more homogeneous populations of cells.

The cells of the pharmaceutical composition may be isolated from an animal before, concurrently and/or after administration of G-CSF, G-CSF derivative or G-CSF mimetic to the animal as described herein. The isolated cells may be cultured in vitro in the presence of one or more actives, including IL-10, GM-CSF and/or TGF-β as described herein. Culturing of the isolated cells may enrich or purify a population of cells from a heterogeneous cell population to thereby result in a homogeneous or substantially homogeneous population of cells, for example a homogeneous population of T cells and/or GM cells. Culturing of the isolated cells in one embodiment propagates the isolated cells to thereby increase a total number of cells. Preferably, the isolated cells are human cells.

Suitably, the pharmaceutical composition comprises a pharmaceutically-acceptable carrier. By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including phosphate buffered solutions, sugars, starches, cellulose and its derivatives, malt, gelatine, talc, calcium sulfate, vegetable oils, synthetic oils, polyols, alginic acid, emulsifiers, isotonic saline, and pyrogen-free water.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. An example of a suitable dosage form is Neulasta™, which is supplied from Amgen Inc as a preservative-free solution comprising 6 mg (0.6 mL) of pegfilgrastim (10 mg/mL) in a single-dose syringe with a 27 gauge, ½ inch needle. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Delivery of Pharmaceutical Compositions of the Invention

Any suitable route of administration may be used for providing an individual with the composition of the invention. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed.

Preferably, the G-CSF derivative is administered by subcutaneous injection of the donor.

A preferred form of administration of the pharmaceutical composition comprising cells treated in accordance with the methods of the invention is by intravenous injection. However, other routes of administration may be used as described above. The pharmaceutical composition in one embodiment is administered to a recipient at a site of solid organ transplantation during transplantation. The composition of the invention may further include any other suitable agent, for example an antibiotic, immune suppressing agent, cytokine or any other agent selected by a skilled person that may assist in preventing GVHD and improve survival and recovery of the recipient.

The cells of the composition in one embodiment are propagated in vitro to increase cell number as described above before transplantation.

The cells of the composition may be treated in vivo by administration of G-CSF derivative to the donor before transplantation of donor cells to the recipient. It will be appreciated that in addition to treatment with the G-CSF derivative, the cells of the composition may further be treated with G-CSF and/or a G-CSF mimetic either in vivo and/or in vitro. A suitable route of administration may be selected by a person skilled in the art, including routes described above. Administration may be via dosage forms as described hereinafter. The cells of the composition may be treated in vitro by exposure to G-CSF, G-CSF derivative or G-CSF mimetic, for example addition of G-CSF, G-CSF derivative or G-CSF mimetic to cell culture media during cell culturing. It will be appreciated that alternatively, or in addition to the above agents, other suitable growth factors, cytokines, antibiotics and agents may be added to the culture media to improve propagation and cell survival. Cells treated in accordance with the invention suitably produce IL-10. IL-10 may be measured using well known methods as herein described including by ELISA using antibodies specific for IL-10. Such antibodies may be monoclonal or polyclonal.

Compositions of the present invention suitable for administration may be presented as discrete units such as vials, capsules, sachets or tablets each containing a pre-determined amount of one or more immunogenic agent of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more immunogenic agents as described above with the carrier which constitutes one or more necessary ingredients. In general, the compositions are prepared by uniformly and intimately admixing the agents of the invention with liquid carriers or finely divided solid carriers or both, and then, if necessary, shaping the product into the desired presentation.

In a preferred form of the invention, the pharmaceutical composition comprising a G-CSF derivative is administered to a subject, preferably a human patient, as a single dose. However, it will be appreciated that the pharmaceutical composition may be administered in several same or differing doses, for example two, three, four, five, six, seven, eight, nine, ten, or more doses.

Preferably dosage ranges for administering a G-CSF derivative, preferably peg-G-CSF, include between 60 μg/kg (body weight of subject)—300 μg/kg, 75 μg/kg -250 μg/kg, 100 μg/kg -225 μg/kg, 125 μg/kg -175 μg/kg and 150 μg/kg -200 μg/kg. It will be appreciated that any value with the indicated ranges may be preferably used, for example, 60 μg/kg, 80 μg/kg, 100 μg/kg, 125 μg/kg, 150 μg/kg, 175 μg/kg, 200 μg/kg, 250 μg/kg, 300 μg/kg and any other value between the above ranges. Preferably, for an average human adult (preferably weighing more than 45 kg) about 6 mg-18 mg of the G-CSF derivative, preferably peg-G-CSF, is administered, preferably as a single subcutaneous injection. Other suitable ranges include 2 mg-30 mg, 5 mg-20 mg and 10 mg-15 mg. For Neulasta™, a preferred dosage is 6 mg for a human, preferably a human weighing more than 45 Kg. A preferred subject is a patient, preferably the patient is a human patient. The human patient is preferably a donor in relation to inducing transplantation tolerance and in relation to self-tolerance the human patient is preferably predisposed or presenting with an autoimmune disorder. Preferably, the patient is predisposed and asymptomatic for an autoimmune disorder.

Transplantation

It will be appreciated that although the experiments described herein describe transplantation of spleen derived stem cells to a recipient, other cell types may be transplanted. For example, stem cells isolated from blood, bone marrow, skin, hair follicle, neuronal tissue or any other suitable source. The invention may also be used in relation to solid organ transplantation, such as transplantation of heart, lung, liver, kidney, skin or any other suitable organ, tissue or part thereof. Methods for transplantation are well known any suitable transplantation method may be used in accordance with the invention. Also, transplantation refers to heterologous transplantation of cells, tissue or organ from a different donor than the recipient. Autologous transplantation encompasses isolating a cell, tissue or organ from a donor and transplanting the isolated cell, tissue or organ into a recipient, who is the donor. For example, cells of a human patient predisposed to an autoimmune disorder may be isolated, preferably T cells and/or GM cells, administered with a G-CSF derivative or biologically active fragment, homolog or variant thereof and transplanted back to the same human patient. The isolated cells are preferably propagated in vitro before transplanting back into the human patient.

“Graft versus host disease” (GVHD) also refers to “graft versus host reaction” meaning, a reaction wherein immunocompetent cells from a donor transplant immunologically react with antigens of the recipient. GVHD typically occurs following allogeneic stem cell transplantation due to HLA disparity between donor and recipient. Donor T cells treated in accordance with the invention are particularly useful in preventing or reducing GVHD, thereby improving recovery and survival of the recipient. It will understood that the invention does not need to totally prevent GVHD or completely render an animal immunologically tolerant to be useful and that partial tolerance or extended period of survival is also useful.

“Tolerance” also refers to “immunological tolerance, immuno-tolerance and non-responder tolerance” meaning a decrease in, or loss of, an ability of an animal to produce an immune response upon administration of an antigen. Theories of tolerance induction include clonal deletion and clonal anergy. In clonal deletion, the actual clone of cells is eliminated whereas in clonal anergy the cells are present, but are immunologically nonfunctional. Tolerance may also refer to a decrease in, or loss of, an ability of immuno-competent cells from a donor to produce an immune response, for example a decrease in, or loss of, GVHD. A preferred embodiment of the present invention relates to a method for inducing transplantation tolerance, however, it will be appreciated that another preferred embodiment of the invention relates to preventing, treating or improving a condition of a patient in relation to an autoimmune disorder wherein transplantation is not required and may be omitted.

An embodiment of the invention wherein an autoimmune disorder is prevented or onset reduced, physical symptoms of the autoimmune disorder may not be present (asymptomatic) in the subject, preferably a human patient. For such an embodiment, a predisposition to an onset of an autoimmune disorder may be determined by genetic or family history, medical examination, correlation with one or more markers, including genetic/molecular markers (for example methods involving marker assessment using RFLP, AFLP, microarrays and like methods as are commonly known in the art). Non-limiting examples of autoimmune disorders include rheumatoid arthritis, systemic lupus erythematosus, multiple sclerosis and inflammatory bowel disease.

In one form of the invention, a CSF derivative or biologically active fragment, homolog, variant or G-CSF mimetic is preferably administered to the patient by injection. Not being bound by theory, administering the G-CSF derivative or G-CSF mimetic may activate T cells directly to produce IL-10 and/or activate granulocyte-monocytes of the donor to thereby stimulate IL-10 secreting T cells as described herein. The granulocyte-monocytes in one form of the invention may be propagated in vitro to thereby increase the number of cells capable of stimulating IL-10 secreting T cells. Likewise, donor T cells may be propagated as a mixed culture or separate culture.

Inducing immunological tolerance, in particular transplantation tolerance, preferably results in an increase in survival or an improvement in a medical condition of a patient. Inducing immunological tolerance need to completely prevent GVHD to be useful and partial prevention of GVHD in one embodiment of the invention is beneficial to the patient or recipient.

The present invention may be particularly useful with allogeneic transplantation, for example allogeneic stem cell, tissue or organ transplantation, because allogeneic transplantation typically results in GVHD. “Allogeneic transplantation” refers to transplantation of a cell, organ or tissue, or part thereof, that is donated either by a genetically matched donor such as a relative of the patient or by an unrelated (but often genetically similar) donor. Two or more individuals are considered to be allogeneic to one another when the genes at one or more loci are not identical in sequence in each organism. It will be appreciated that the invention may also be used in relation to syngneic transplantation.

The donor and recipient of a transplant are preferably mammals, including for example humans, primates, livestock (eg cattle, sheep, pigs), race animals (eg horse, dog, camel), domesticated companion animals (eg dogs, cats) and research animals (eg mice, rats, rabbits, goats). The mammal is preferably human, for example a human patient. Preferably, the donor and recipient are the same species, although transplantation between species, ie xenotransplantation, falls within the scope of transplantation.

The pharmaceutical composition comprising cells as describe herein are preferably used in transplantation to prevent or reduce GVHD. The cells treated in accordance with the invention are preferably transplanted at the same time as other cells, for example stem cells or solid tissue or organ. However, the cells treated in accordance with the invention may be transplanted before, simultaneously (eg co-administered) and/or after transplantation of other cells. A single pharmaceutical composition may comprise a plurality of cells to be simultaneously transplanted. Alternatively, a plurality of cells may be simultaneously transplanted by simultaneous administration of two or more pharmaceutical compositions (for example two, three, four, five, six, seven, eight, nine, ten or more), each comprising one or more cell types (for example, one, two, three, four, five, six, seven, eight, nine, ten or more cell types).

In order that the invention may be readily understood and put into practical effect, particular preferred embodiments will now be described by way of the following non-limiting examples.

### EXAMPLE 1

Methods

Mice.

Female C57BL/6 (B6, H-2b, Ly-5.2+), B6 PTRCA Ly-5a (H-2b, Ly-5.1+) and B6D2F1 (H-2b/d, Ly-5.2+) (Morse et al, 1987) mice were purchased from the Australian Research Centre (Perth, Western Australia, Australia). C57BL/6 IL-10^{-/-} mice (B6, H-2b, Ly-5.2+) supplied by the Australian National University (Canberra, Australia). The age of mice used as BMT recipients ranged between 8 and 14 weeks. Mice were housed in sterilised microisolator cages and received acidified autoclaved water (pH 2.5) and normal chow for the first two weeks post BMT.

Cytokine treatment.

Murine G-CSF (Amgen, Thousand Oaks, Calif., USA), recombinant human G-CSF (Amgen, Thousand Oaks, Calif., USA), pegylated recombinant human G-CSF (peg-G-CSF) (Amgen, Thousand Oaks, Calif., USA) or control diluent was diluted in 1 ug/ml of murine serum albumin in PBS before injection. Mice were injected subcutaneously with doses of murine or human G-CSF from days —6 to —1, or peg-G-CSF on day —6 at doses as stated.

Stem Cell Transplantation.

Mice were transplanted according to a standard protocol as has been described previously (Pan et al, 1995; Pan et al, 1999), both incorporated herein by reference. Briefly, on day —1, B6D2F1 mice received 1100 cGy total body irradiation (137Cs source at 108 cGy/min), split into two doses separated by 3 hours to minimise gastrointestinal toxicity. Donor splenocytes resuspended in 0.25 ml of Leibovitz's L-15 media (Gibco BRL, Gaithersburg Md.) were injected intravenously into recipients. T cell depletion (via 2 cycles of anti-CD4, anti-CD8 and anti-Thy1.2 plus rabbit complement) or T cell purification (via teased nylon wool column purification) were performed as indicated. Survival was monitored daily, and GVHD clinical score were measured weekly.

Assessment of GVHD.

The degree of systemic GVHD was assessed by a scoring system which sums changes in five clinical parameters: weight loss, posture (hunching), activity, fur texture and skin integrity (maximum index=10) (Hill et al, 1997; Hill et al, 1998; Hill et al, 1999). Individual mice were ear-tagged and graded weekly from 0 to 2 for each criterion without knowledge of treatment group. Animals with severe clinical GVHD (scores>6) were sacrificed according to ethical guidelines and the day of death deemed to be the following day.

Cell preparation.

DC purification was undertaken as previously described (1). Briefly, low-density cells were selected from digested spleen by nycodenz density gradient (1.077 g/l) centrifugation. Non DC-lineage cells were depleted by coating with rat IgG antibodies to B cells (CD19), T cells (CD3, Thy1), granulocytes (Gr-1) and erythroid cells (Ter-119). The coated cells were then removed by magnetic beads coupled to anti-rat IgG (Dynal ASA, Oslo, Norway). At the end of this procedure, 50-70% of these cell populations were DC (class II⁺/CD11c⁺) and 30-50% were GM cells. GM were FACS sorted (Moflo, Dako-Cytomation, Colo., USA) as the negative staining population following staining with CD11c-FITC and PE-conjugated lineage antibodies (B220, CD19, CD3). At the end of

FACS analysis.

Fluorescein isothiocyanate (FITC) conjugated monoclonal antibodies (mAb) CD3, CD4, CD8, CD11b, CD11c, class II, B220 and identical phycoerythrin (PE) conjugated antibodies were purchased from PharMingen (San Diego, Calif., USA). Cells were first incubated with mAb 2.4 G2 for 15 minutes at 4° C., then with the relevant conjugated mAb for 30 minutes at 4° C. Finally, cells were washed twice with PBS/0.2% BSA, fixed with PBS/1% paraformaldehyde and analysed by FACScan (Becton Dickinson, San Jose, Calif., USA).

Cell cultures.

Culture media additives were purchased from Gibco BRL (Gaithersburg, Md., USA) and media was purchased from Sigma (St Louis, Mo., USA). Cell culture was performed in 10% FCS/RPMI supplemented with, 50 units/ml penicillin, 50 μg/ml streptomycin, 2 mM L-glutamine, 1 mM sodium pyruvate, 0.1 mM non-essential amino acid, 0.02 mM β-mercaptoethanol, and 10 mM HEPES, pH 7.75 at 37° C. in a humidified incubator supplemented with 5% CO2. For in vitro allo-antigen experiments, purified B6 T cells were cultured in round bottom 96 well plates (Falcon, Lincoln park, N.J., USA) with 105 irradiated (2000 cGy) F1 peritoneal macrophages (primary MLC) and supernatants harvested at 72 hours. Cultures were then pulsed with 3H-thymidine (1 μCi per well) and proliferation was determined 16 hrs later on a 1205 Betaplate reader (Wallac, Turku, Finland). For in vitro mitogen stimulation, purified B6 T cells were cultured in flat bottomed 96 well plates, pre-coated with monoclonal CD3 and CD28 at final concentrations of 10 μg/ml. Supernatants were harvested at 48 hours and cultures pulsed with 3H-thymidine (1 μCi per well). Proliferation was determined 16 hrs later on a 1205 Betaplate reader (Wallac, Turku, Finland). In secondary MLC, purified T cells were cultured in flat bottom 24 well plates (Falcon, Lincoln park, N.J., USA) with irradiated (2000 cGy) splenocytes. Six days later, cells were removed and restimulated with F1 macrophages. Supernatants were removed 24 hrs later and 3H-thymidine added as above.

Transplantation.

Mice were transplanted according to a standard protocol as has been described previously (1). Briefly, on day —1, B6D2F1 mice received 1100 cGy total body irradiation (137 Cs source at 108 cGy/min), split into two doses separated by 3 hours. Donor spleens were chopped, digested in collagenase and DNAse, then whole unseparated spleen cells (10⁷ splenocytes unless otherwise stated) injected intravenously into recipients. In one group, purified GM cells (10⁶) were added to the control splenocytes. Survival was monitored daily, recipient's body weights and GVHD clinical score were measured weekly.

Cytokine ELISAS.

The antibodies used in the TNFα, IFNγ, IL-10 and IL-4 assays were purchased from PharMingen (San Diego, Calif., USA). All assays were performed according to the manufacturer's protocol. Briefly, samples were diluted 1:3 to 1:24 and TNFα, IFNγ, IL-10 and IL-4 proteins were captured by the specific primary monoclonal antibody (mAb), and detected by biotin-labelled secondary mAbs. The biotin-labelled assays were developed with strepavidin and substrate (Kirkegaard and Perry laboratories, Gaithersburg, Md., USA). Plates were read at 450 nm using a microplate reader (Bio-Rad Labs, Hercules, Calif., USA). Recombinant cytokines (PharMingen) were used as standards for ELISA assays. Samples and standards were run in duplicate and the sensitivity of the assays was 16 to 20 pg/ml for TNFα, 0.063 U/ml for IFNγ, and 15 pg/ml for IL-10 and IL-4. Supernatants were collected after 4 hours of culture for TNFα 40 hours for IL-4, IL-10 and IFNγ analysis. Serum was stored at −70 C. until analysis.

Statistical analysis.

Survival curves were plotted using Kaplan-Meier estimates and compared by log-rank analysis. The Mann Whitney-U test was used for the statistical analysis of cytokine data and clinical scores. P<0.05 was considered statistically significant.

### EXAMPLE 2

Donor Pre-Treatment with Recombinant Human G-CSF Prevents GVHD in a Dose-Dependant Fashion

The present investigators examined the effect of incrementally increasing the dose of G-CSF administered to SCT donors in a well-established murine SCT model (C57BL/6 Ly5^{a}→B6D2F1) that induces GVHD to major and minor histocompatibility antigens. Although this model utilises spleen as a stem cell source rather than peripheral blood, it's validity has been proven by informative data indicating beneficial effects of G-CSF on both GVHD and GVL (Pan et al, 1995; Pan et al, 1999) that have since been confirmed clinically (Bensinger et al, 2001). Allogeneic donor C57BL/6 animals received 6 daily injections of either control diluent, 0.2 μg human G-CSF, 2 μg human G-CSF or 10 μg human G-CSF and spleens were harvested on day 7. B6D2F1 recipient mice received 1100 cGy of TBI, and splenocytes (corrected to administer 3×10⁶ T cells per inoculum) transplanted intravenously from respective donors the following day. As shown in FIG. I a, GVHD induced in this model is severe with all recipients of control splenocytes dying within two weeks with characteristic features of GVHD (weight loss, hunching, fur ruffling, etc). In contrast, 100% of non-GVHD controls transplanted with syngeneic splenocytes survived, confirming that this splenocyte dose contained sufficient stem cells to rescue lethally irradiated recipients. Donor pre-treatment with 0.2 μg, 2.0 μg or 10.0 μg of human G-CSF per day for six days resulted in dose-dependant protection from GVHD lethality, with allogeneic SCT recipient survival at day +60 of 0%, 11% or 50% respectively (P<0.05). Clinical GVHD, assessed by clinical in surviving animals, demonstrated that G-CSF did not completely prevent GVHD, but donor pre-treatment with G-CSF 10 μg/day provides greater protection than mobilisation with 2 μg/day or 0.2 μg/day (P<0.05).

### EXAMPLE 3

Donor Pre-Treatment with Murine G-CSF Provides Equivalent Protection to Human G-CSF from GVHD at a 10-Fold Lower Dose

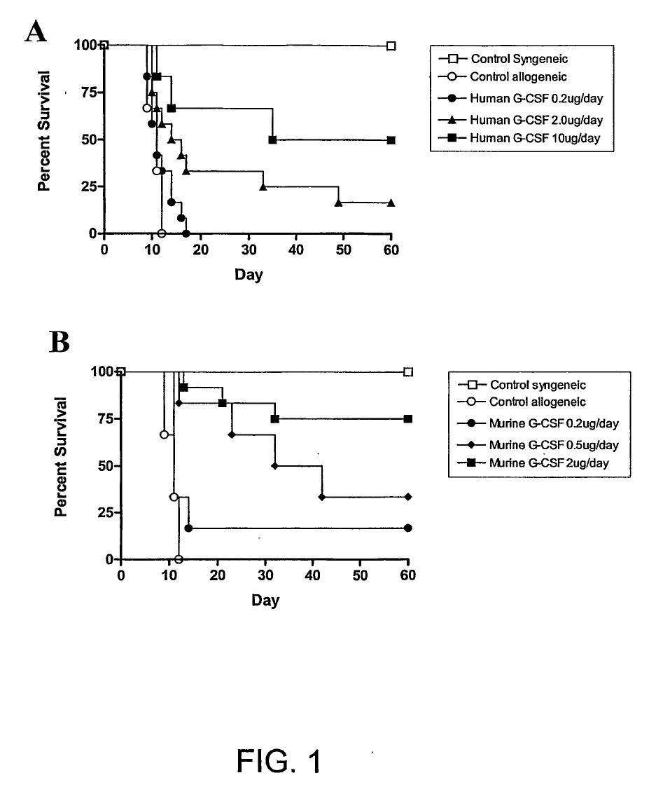

The present investigators sought to determine the relative efficacy of murine G-CSF to prevent GVHD compared to human G-CSF. Allogeneic donor C57BL/6 animals received 6 daily injections of either control diluent, 0.2 μg murine G-CSF, 0.5 μg murine G-CSF or 2 μg murine G-CSF. As shown in FIG. 1b, donor pre-treatment with 0.2 μg, 0.5 μg or 2 μg of murine G-CSF again provided dose dependant protection from GVHD lethality, with survival at day 60 of 17%, 33% or 75% respectively (P<0.05). Survival at day 60 for recipients of splenocytes pre-treated with 0.2 μg of murine G-CSF was equivalent to recipients of splenocytes pre-treated with a ten-fold higher dose of human G-CSF (0.2 μg murine G-CSF day 60 survival 17% versus 2.0 μg human G-CSF day 60 survival 11%, P=0.63).

### EXAMPLE 4

Donor Pre-Treatment with Peg-G-CSF Is Markedly Superior to Standard G-CSF in Preventing Graft-Versus-Host Disease

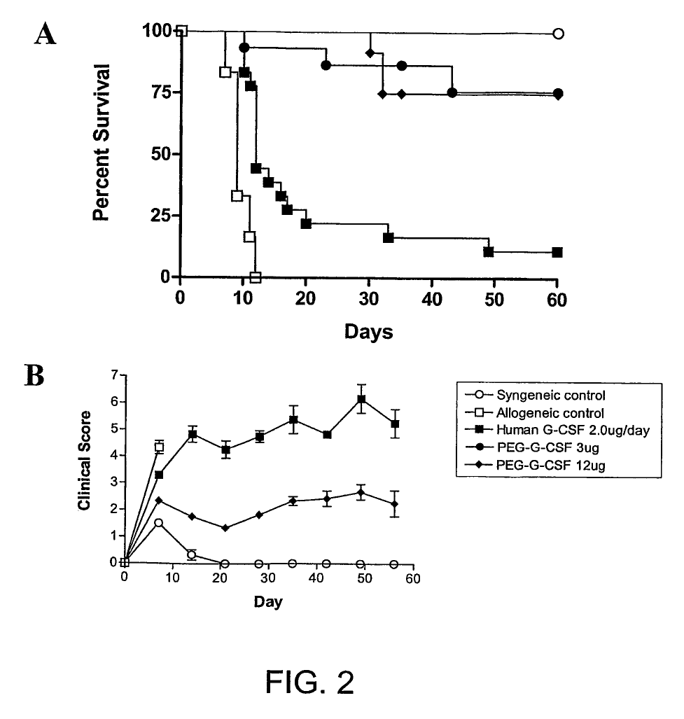

The present investigators next examined whether the increase in plasma half-life attributable to pegylation of G-CSF led to increased protection from GVHD. Allogeneic donor C57BL/6 animals received either control diluent, 2 μg/day for 6 days of standard G-CSF, or a single dose of peg-G-CSF (3 or 12 μg) at day —6. Lethally irradiated B6D2F1 recipient mice were transplanted as above, and grafts were normalised to contain equal numbers of T cells. As shown in FIG. 2a, donor pre-treatment with 3 μg or 12 μg peg-G-CSF resulted in 83% recipient survival at day 60. Donor pre-treatment with 12 μg peg-G-CSF provides significantly more protection from GVHD lethality than the same dose of “standard” human G-CSF given over 6 days (P<0.0001). GVHD clinical scores (weight loss, hunching, fur ruffling, etc) were significantly lower in recipients of peg-G-CSF pre-treated spleen compared with recipients of G-CSF treated splenocytes (P<0.05 at time points as shown FIG. 2b). In addition, histological examination was perform on liver, skin and bowel of surviving animals receiving grafts from donors pre-treated with peg-G-CSF (data not shown).

### EXAMPLE 5

Cellular Expansion Following Donor Pre-Treatment with Standard and Peg-G-CSF

G-CSF has been shown to alter APC phenotype in stem cell grafts, and the present investigators have shown that this contributes to the attenuation of GVHD. We therefore examined both overall spleen expansion and cellular composition following G-CSF or peg-G-CSF pre-treatment. Donor pre-treatment with 2 μg per day of standard G-CSF for 6 days lead to an average 53% increase in spleen size (control versus 2 μg/day G-CSF for 6 days P<0.0001). Pretreatment with a single dose of 12 μg peg-G-CSF lead to an average 65% increase in spleen size (control versus 12 μg peg-G-CSF day —7 P<0.0001). The difference in spleen size between 2 μg G-CSF for 6 days and 12 μg peg-G-CSF as a single dose was not statistically significant (P=0.11).

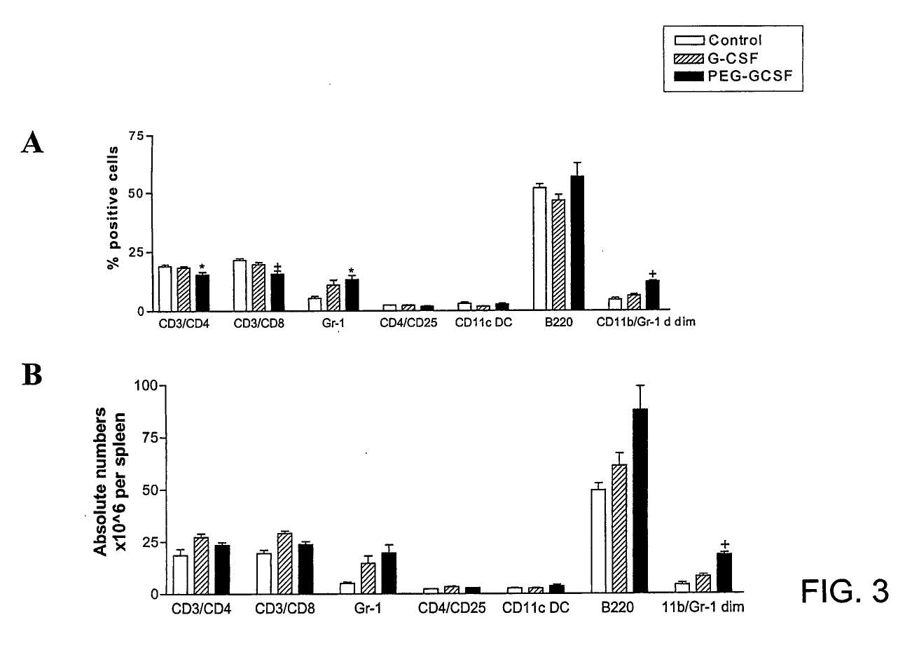

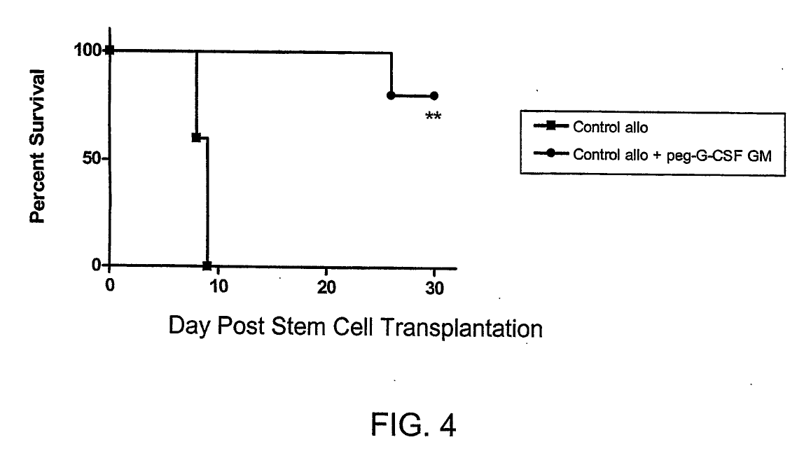

Pre-treatment with 12 μg peg-G-CSF did not alter the total T cell number or sub-set proportions, and in particular the numbers of CD11c⁺DC and CD4+CD25+ regulatory T cells were not altered (FIGS. 3a and 3b). The granulocyte lineage was expanded twofold in peg-G-CSF treated spleens and bone marrow, and to a lesser degree in G-CSF treated spleens (data not shown). As shown in FIGS. 3a and 3b, a novel population of GM cells, defined by a CD11b^{pos}/Gr-1^{dim} phenotype, were disproportionately increased relative to other APC subsets in peg-G-CSF treated donors (G-CSF versus peg-G-CSF P=0.001). Preferably, these GM cells are also CD11c negative. FIG. 4 shows percent survival of animals administered with the abovementioned GM cell population characterised by a CD11^{pos}/Gr-1^{dim} phenotype. The FACS isolated GM cells were capable of preventing GVHD when compared with controls and this protection was due to the expansion of CD4+IL-10 producing regulatory T cells. Thus the treatment of stem cell transplant donors with peg-G-CSF expands or modifies a myeloid and T cell population that promote tolerance.

### EXAMPLE 6

Donor Treatment with Peg-G-CSF Impairs T Cell Function and Induces Regulatory T Cell Activity

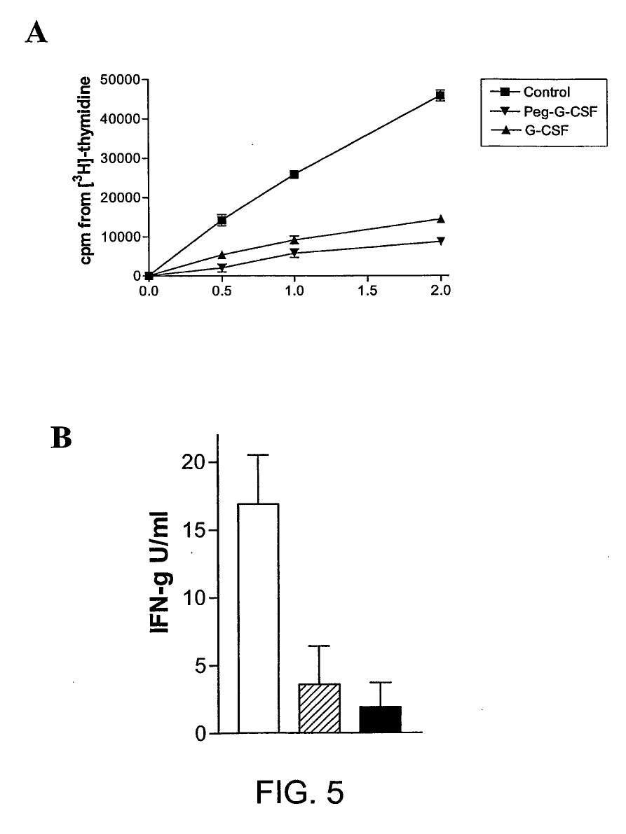

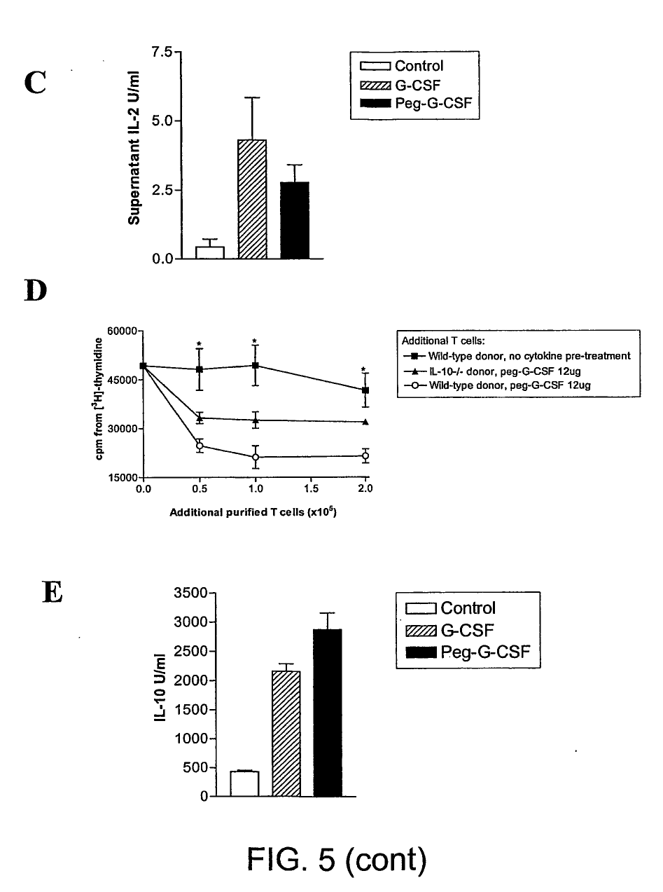

GVHD induced in these models is dependant on T cell function, (Pan et al, 1999; Teshima et al, 1999) and we therefore examined the effect of G-CSF and peg-G-CSF on T cell function in vitro. C57BL/6 T cells were stimulated with alloantigen and T cell proliferation and cytokine production was determined. Pre-treatment of donors with both G-CSF and peg-G-CSF inhibited T cell proliferation to alloantigen, but did not prevent IL-2 production (FIG. 5a). Interferon-γ secretion to alloantigen was reduced 10-fold following donor treatment with peg-G-CSF. Donor T cells from peg-G-CSF animals in response to mitogen (CD3 and CD28) were also reduced 10-fold both pre and post transplant relative to T cells from control treated donors (data not shown). Since the impairment of T cell proliferation was not associated with reductions in IL-2 production, the investigators next sought to determine whether T cells from cytokine pre-treated donors exhibited regulatory function and were able to inhibit the proliferation of T cells from control treated donors. T cells from non-cytokine exposed C57BL/6 donors were stimulated with alloantigen, with or without the addition of T cells from wild-type of IL-10^{-/-} donors, pre-treated with a single dose (12 μg) of peg-G-CSF. As shown in FIG. 5b, T cells from peg-G-CSF pre-treated wild-type donors markedly reduced proliferation (P<0.05 at all T cell doses as shown). T cells from IL-10^{-/-} donors impaired proliferation, but to a lesser degree (FIG. 5b) suggesting that IL-10 production is required by donor T cells, at least in part, to provide a regulatory function. Since IL-10 appeared to be playing a role in the inhibition of T cell function from peg-G-CSF treated donors in vitro, the investigators next studied an ability of grafts from these animals to produce IL-10 in response to inflammatory stimuli. Surprisingly, spleen from both G-CSF and peg-GCSF treated donors produced 8-fold more IL-10 in response to LPS and CPG relative to control treated spleen (FIG. 5c).

### EXAMPLE 7

The Protection from GVHD Is Dependent on Production of IL-10 from the Donor T Cell

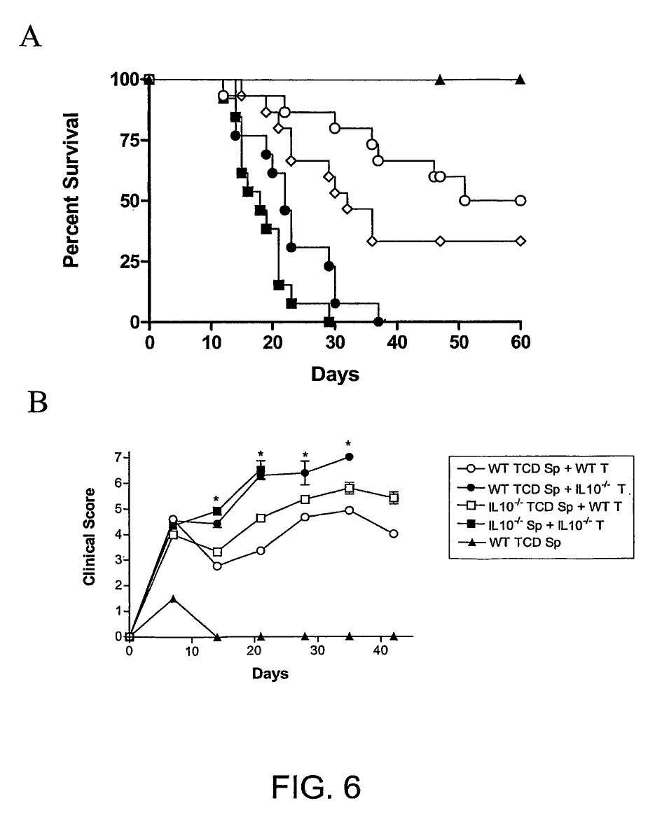

Splenocytes pre-treated with peg-G-CSF produced large amounts of IL-10 in response to inflammatory stimuli and T cells from peg-G-CSF pre-treated donors regulated proliferation of allo-antigen stimulated T cells in vitro in an IL-10 dependent fashion. The investigators therefore next examined whether the protection from GVHD afforded by peg-G-CSF was dependant on IL-10 production by the donor T cell, the non-T cell compartment, or both. C57BL/6 donors in which the IL-10 gene has been homologously deleted (IL-10^{-/-}) were pre-treated with 12 μg peg-G-CSF on day —6. Wild type T cell depleted (TCD) splenocytes from non-cytokine pre-treated donors plus purified T cells from either wild-type or IL-10^{-/-} donors were infused into lethally irradiated B6D2F1 recipients (FIGS. 12a and 12b). Survival at day 60 was 100% in recipients of wild-type TCD and IL-10^{-/-} TCD spleen alone, confirming that adequate numbers of stem cells were transferred to allow haemopoietic reconstitution. Recipients of allogeneic wild-type T cells had delayed mortality (FIG. 6a) and moderate GVHD as assessed by clinical scores (FIG. 6b), regardless of whether the non-T cell component was from wild-type or IL-10^{-/-} donors. In contrast, recipients of allogeneic IL10^{-/-} T cells all died from GVHD by day 30 regardless of whether the non-T cell component was from wild-type of IL-10^{-/-} donors. Thus, the production of IL-10 by donor T cells is causally associated with protection from GVHD afforded by donors pre-treated with peg-G-CSF. In contrast, IL-10 production by the non-T cell compartment did not influence GVHD.

### EXAMPLE 8

The IL-10 Producing Protective Donor T Cell has Regulatory Function

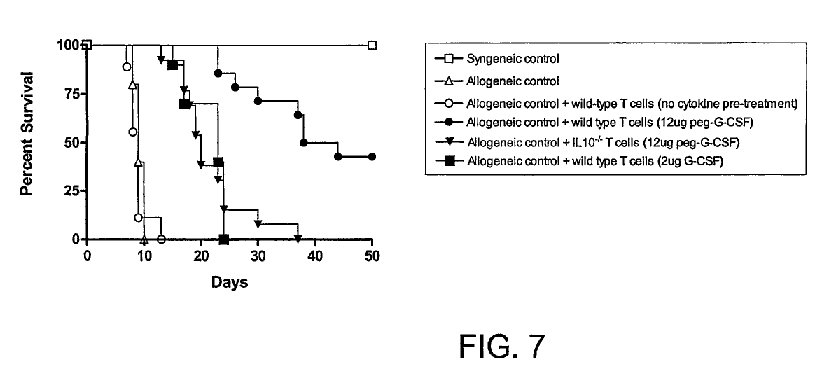

Since the protection from GVHD afforded by peg-G-CSF administration was dependent on IL-10 production by the donor T cell, the present investigators next studied whether these T cells were able to induce infectious tolerance. T cells from wild-type donors pre-treated with control diluent or peg-G-CSF, or IL-10^{-/-} donors pre-treated with peg-G-CSF, were added to wild-type T cell replete grafts from untreated donors. As shown in FIG. 7, the addition of T cells from control treated donors to control grafts did not prevent GVHD mortality with all animals dying by day 12. In contrast, the addition of T cells from peg-G-CSF treated donors to control grafts resulted in 45% survival at day 50 (P<0.001). This ability to regulate GVHD was significantly greater in T cells from peg-G-CSF treated donors compared to donor T cells from standard G-CSF treated donors since the later provided only a modest 10 day delay in mortality. The regulation of GVHD by T cells from peg-GSF treated donors was largely, although not completely, dependant on IL-10 production by the donor T cell, since T cells from peg-G-CSF pre-treated IL-10^{-/-} donors delayed, but did not prevent GVHD mortality.

### EXAMPLE 9

Peg-G-CSF Is an Effective Agent for the Mobilisation and Collection of Allogeneic Stem Cells for Transplantation and Prevention of GVHD

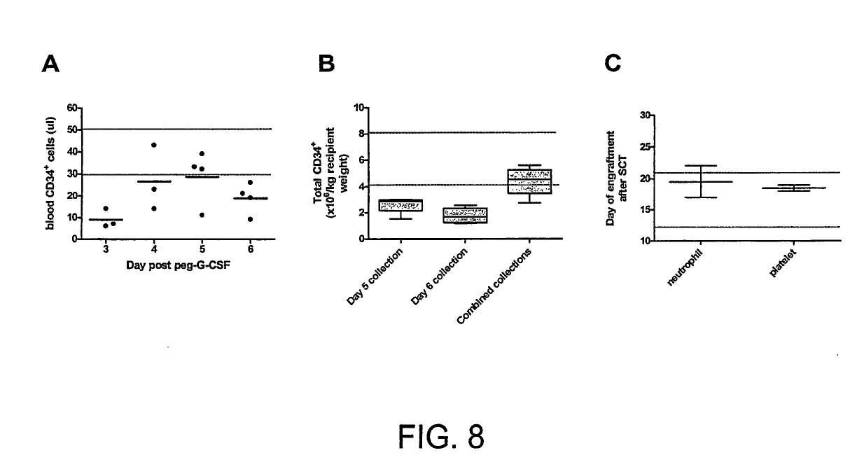

Human Patients: FIG. 8 shows data in relation to human patients. Patients receiving myeloablative allogeneic stem cell transplants and their HLA compatible siblings were enrolled on the institutionally approved protocol following signed informed consent. Donors received 6 mg of pegylated-G-CSF (Neulasta™, Amgen, Thousand Oaks, Calif.) as a single dose and CD34⁺ counts determined in the peripheral blood 3-6 days later as previously described (2). Donors were then underwent 1.5 blood volume aphaeresis on day 5 and 6 and the total numbers of CD34 cells (per kg of recipient weight) enumerated by standard flow cytometry (2). The products were combined and transfused fresh into transplant recipients who had received myeloablative conditioning with total body irradiation and cyclophosphamide. The day of neutrophil recovery was determined as the first day >0.5×10⁹/L. The day of platelet recovery was determined as the first of 5 days in which the unsupported platelet count was >20×10⁹/L.

Experimental data has been collected from a phase I/II clinical trial to study an ability of peg-G-CSF to mobilise stem cells and subsequent ability to restore haematopoiesis after transplantation, as shown in FIGS. 8A-8C. Human clinical trial data shows that some human patients administered peg-G-CSF have thus far not presented with symptoms of GVHD. This data also shows that for all normal sibling donors under 100 kg a single administration of a 6 mg dose of peg-G-CSF (n=4) that represented 79±3 μg/kg of peg-G-CSF may be sufficient.

Donors experienced only minor side effects from the administration of peg-G-CSF that were similar to that seen with standard G-CSF administration. These data confirm the feasibility of performing allogeneic stem cell transplantation with stem cells mobilised by peg-G-CSF and use of peg-G-CSF for preventing or reducing an occurrence of GVHD.

### EXAMPLE 10

Peg-G-CSF Administration for Inducing Self-Tolerance

A patient is administered peg-G-CSF as described above for preventing or reducing GVHD. Peg-G-CSF is administered in a range of 60 μg/kg-300 μg/kg total weigh, but preferably, 6 mg of peg-G-CSF is administered to the patient, wherein the patient is a human weighing more than 45 Kg. The peg-G-CSF is administered as a single subcutaneous injection, however, multiple injections may be administered, for example, two, three, four, five, six, seven, eight, nine, ten or more doses. Multiple doses may be required to prevent onset of an autoimmune disorder. The peg-G-CSF is available from Amgen, Inc sold under the trade name, Neulasta™. A pharmaceutical composition comprising peg-G-CSF or biological fragment, homolog or variant thereof similar to Neulasta™ may likewise be used.

In a preferred embodiment, peg-G-CSF is administered to a patient not yet showing symptoms of an autoimmune disorder to prevent onset of one or more autoimmune disorders. Predisposition of the patient to an autoimmune disorder may be determined by reviewing family history for an occurrence of an autoimmune disorder, exposure to environmental conditions that may result in an autoimmune disorder, genetic testing for a molecular marker correlated with one or more autoimmune disorder and the like. Genetic testing may include use of methods such as AFLP, RFLP and use of microarray technology wherein one or more target nucleic acids correlated with one or more autoimmune disorders are located on a microarrary chip and binding by a nucleic acid of the patient is determined. The nucleic acid from the patient may be isolated from any suitable source from the patient, including a biological sample from blood, tissue, organ or body fluid.

### EXAMPLE 11

Discussion

The present investigators show that donor pre-treatment with recombinant human G-CSF protects recipients from GVHD in a dose dependant fashion. Also, treatment of mice with murine G-CSF is approximately 10-fold more potent than human G-CSF, indicating that G-CSF comprising an amino acid sequence that is the same as or similar to that of the donor species is preferred. For example, human G-CSF (including peg-human-G-CSF, peg-recombinant human-G-CSF) for use with human donor and recipients. In addition, donor pre-treatment with a single dose of peg-G-CSF significantly reduces GVHD when compared with the same dose of standard G-CSF given over 6 days. It will be appreciated that although the example demonstrate that a single dose of peg-G-CSF is suitable, the invention contemplates more than one dose or administration of peg-G-CSF, for example, two, three, four, five, six, seven, eight, nine, ten or more doses. The protection from GVHD appears to be dependant on donor T cell production of IL-10, and T cells from cytokine pre-treated donors have transferable regulatory activity both in vivo.

Species-specific G-CSF (i.e. murine G-CSF in murine transplants) was able to confer equivalent GVHD protection at a 10-fold lower dose than human G-CSF in a murine model. Not being bound by theory, this is likely to reflect superior ligand-receptor interaction between murine G-CSF and the murine G-CSF than between human G-CSF and murine G-CSF receptors. Pegylation of G-CSF significantly increases the plasma half-life of G-CSF, without altering receptor affinity. Thus, not being bound by theory, increased receptor occupancy over a prolonged period leads to further increases in therapeutic efficacy, with significantly improved survival of animals receiving splenocytes from donors pre-treated with a single dose of peg-G-CSF, compared with recipients receiving splenocytes from donors pre-treated with the same dose of standard G-CSF over 6 days.

The present investigators demonstrate that peg-G-CSF leads to an approximate 4-fold expansion of a novel GM APC population, which may be involved with the improvement in regulatory T cell function following donor pre-treatment with peg-G-CSF compared to G-CSF.

CD4⁺CD25⁺ regulatory T cells have been shown to regulate both autoimmune disease (Sakaguchi et al, 1995; Salomon et al, 2000), the rejection of solid organ transplants (Hara et al, 2001) and GVHD (Hoffmann et al, 2002). Cohen and colleagues (Cohen et al, 2002) examined the regulatory effects naturally occurring CD4⁺CD25⁺ T cells (which represent 5-10% of the normal T cell compartment (Levings et al, 2001)) in the B6 to B6D2F1 murine SCT model. They reported that removal of the CD4⁺CD25⁺ T cell compartment from a transplant inoculum resulted in earlier GVHD mortality. Addition of CD4⁺CD25⁺ T cells reduced, although did not prevent, GVHD mortality. Due to the low numbers of CD4⁺CD25⁺ T cells in the peripheral blood healthy donors, stimulation with allogeneic APCs and IL-2 was utilised to induce ex vivo expansion. The CD4⁺CD25⁺ T cells retained their regulatory properties. A significant limitation of this approach, however, was the limited half-life of transferred regulatory T cells, with the dramatic appearance of severe lethal GVHD after only a few weeks. Treatment with peg-G-CSF does not lead to expansion of CD4⁺CD25⁺ T cells, and the regulatory T cell induced by peg-G-CSF in relation to the present invention provide long-lasting transplant tolerance. Thus the protective IL-10 producing T cell does not appear to be a classical CD4⁺CD25⁺ T cell, but is likely to be CD4⁺.

Peg-G-CSF is markedly superior to G-CSF for the long-term prevention of GVHD following allogeneic haematopoietic stem cell transplantation due to the generation of IL-10 producing regulatory donor T cells. These data support the initiation of prospective clinical trials examining the ability of peg-G-CSF mobilised allogeneic peripheral blood stem cell grafts to induce transplant tolerance in both stem cell and solid organ settings. Furthermore, the induction of IL-10 producing regulatory T cells following peg-GCSF administration suggests applicability to a wider variety of diseases characterised by autoimmunity and failure of regulatory tolerance to self antigens.

It is understood that the invention described in detail herein is susceptible to modification and variation, such that embodiments other than those described herein are contemplated which nevertheless falls within the broad scope of the invention.

The disclosure of each patent and scientific document, computer program and algorithm referred to in this specification is incorporated by reference in its entirety.

### REFERENCES

1. Bensinger W I, Martin P J, Storer B, Clift R, Forman S J, Negrin R, Kashyap A, Flowers M E, Lilleby K, Chauncey T R, Storb R, Appelbaum F R. Transplantation of bone marrow as compared with peripheral-blood cells from HLA-identical relatives in patients with hematologic cancers. N Engl J Med. 2001;344:175-181.
2. Pan L, Delmonte J, Jalonen C K, Ferrara J L M. Pretreatment of donors with granulocyte colony-stimulating factor polarizes donor T lymphocytes toward type 2 cytokine production and reduces severity of experimental graft versus host disease. Blood. 1995;86:4422-4429
3. Rutella S, Pierelli L, Bonanno G, Sica S, Ameglio F, Capoluongo E, Mariotti A, Scambia G, d'Onofrio G, Leone G. Role for granulocyte colony-stimulating factor in the generation of human T regulatory type 1 cells. Blood. 2002;100:2562-2571.
4. Streeter P R, Minster N I, Kahn L E, Hood W F, Vickery L E, Thurman T L, Monahan J B, Welply J K, McKearn J P, Woulfe S L. Progenipoietins: biological characterization of a family of dual agonists of fetal liver tyrosine kinase-3 and the granulocyte colony-stimulating factor receptor. Exp Hematol. 2001;29:41-50.
5. Fleming W H, Mulcahy J M, McKearn J P, Streeter P R. Progenipoietin-1: a multifunctional agonist of the granulocyte colony-stimulating factor receptor and fetal liver tyrosine kinase-3 is a potent mobilizer of hematopoietic stem cells. Exp Hematol. 2001;29:943-951.
6. Tayebi H, Kuftler F, Saas P, Lienard A, Petracca B, Lapierre V, Ferrand C, Fest T, Cahn J, Blaise D, Kuentz M, Herve P, Tiberghien P, Robinet E. Effect of granulocyte colony-stimulating factor mobilization on phenotypical and functional properties of immune cells. Exp Hematol. 2001;29:458-470.
7. Ringden O, Labopin M, Bacigalupo A, Arcese W, Schaefer U W, Willemze R, Koc H, Bunjes D, Gluckman E, Rocha V, Schattenberg A, Frassoni F. Transplantation of peripheral blood stem cells as compared with bone marrow from HLA-identical siblings in adult patients with acute myeloid leukaemia and acute lymphoblastic leukaemia. Journal of Clinical Oncology. 2002;20:4655-4664
8. Pan L, Teshima T, Hill G R, Bungard D, Brinson Y S, Reddy V S, Cooke K R, Ferrara J L M. Granulocyte colony-stimulating factor-mobilized allogeneic stem cell transplantation maintains graft-versus-leukemia effects through a perforin-dependent pathway while preventing graft-versus-host disease. Blood. 1999;93:4071-4078
9. Abuchowski A, van Es T, Palczuk N C, Davis F F. Alteration of immunological properties of bovine serum albumin by covalent attachment of polyethylene glycol. J Biol Chem. 1977;252:3578-3581
10. Bailon P, Berthold W. Polyethylene glycol-conjugated pharmaceutical proteins. Pharmaceutical Science and Technology Today. 1998;1:352-356.
11. Molineux G. Pegfilgrastim: using pegylation technology to improve neutropenia support in cancer patients. Anticancer Drugs. 2003;14:259-264
12. Morse H C, Shen F W, Hammerling U. Genetic nomenclature for loci controlling mouse lymphocyte antigens. Immunogenetics. 1987;25:71
13. Hill G R, Crawford J M, Cooke K J, Brinson Y S, Pan L, Ferrara J L M. Total body irradiation and acute graft versus host disease. The role of gastrointestinal damage and inflammatory cytokines. Blood. 1997;90:3204-3213
14. Hill G R, Cooke K R, Teshima T, Crawford J M, Keith J C J, Brinson Y S, Bungard D, Ferrara J L M. lnterleukin-11 promotes T cell polarization and prevents acute graft-versus-host disease after allogeneic bone marrow transplantation. J. Clin. Invest. 1998;102:115-123
15. Hill G R, Teshima T, Gerbita A, Pan L, Cooke K R, Brinson Y S, Crawford J M, Ferrara J L M. Differential Roles of IL-1 and TNFa on Graft-versus-Host Disease and Graft-versus Leukemia. J. Clin. Invest. 1999;104:459-467
16. Teshima T, Hill G R, Pan L, Brinson Y S, van den Brink M R M, Cooke K R, Ferrara J L M. Interleukin-11 improves separates graft-versus-leukemia effects from graft-versus-host disease after bone marrow transplantation. J. Clin. Invest. 1999;104:317-325
17. Sakaguchi S, Sakaguchi N, Asano M, Itoh M, Toda M. Immunologic self-tolerance maintained by activated T cells expressing IL-2 receptor alpha-chains (CD25). Breakdown of a single mechanism of self-tolerance causes various autoimmune diseases. J Immunol. 1995;155:1151-1164.
18. Salomon B, Lenschow D J, Rhee L, Ashourian N, Singh B, Sharpe A, Bluestone J A. B7/CD28 costimulation is essential for the homeostasis of the CD4+CD25+ immunoregulatory T cells that control autoimmune diabetes. Immunity. 2000;12:431-440.
19. Hara M, Kingsley C I, Niimi M, Read S, Turvey S E, Bushell A R, Morris P J, Powrie F, Wood K J. IL-10 is required for regulatory T cells to mediate tolerance to alloantigens in vivo. J Immunol. 2001;166:3789-3796.
20. Hoffmann P, Ermann J, Edinger M, Fathman C G, Strober S. Donor-type CD4(+)CD25(+) regulatory T cells suppress lethal acute graft-versus-host disease after allogeneic bone marrow transplantation. J Exp Med. 2002;196:389-399.
21. Cohen J L, Trenado A, Vasey D, Klatzmann D, Salomon B L. CD4(+)CD25(+) immunoregulatory T Cells: new therapeutics for graft-versus-host disease. J Exp Med. 2002;196:401-406.
22. Levings M K, Sangregorio R, Roncarolo M G. Human cd25(+)cd4(+) t regulatory cells suppress naive and memory T cell proliferation and can be expanded in vitro without loss of function. J Exp Med. 2001;193:1295-1302.
23. MacDonald, K. P., V. Rowe, C. Filippich, R. Thomas, A. D. Clouston, J. K. Welply, D. N. Hart, J. L. Ferrara, and G. R. Hill. 2003. Donor Pretreatment with Progenipoietin-1 is Superior to G-CSF in Preventing Graft-versus-Host Disease after Allogeneic Stem Cell Transplantation. Blood 101:2033.
24. Morton, J., C. Hutchins, and S. Durrant. 2001. Granulocyte-colony-stimulating factor (G-CSF)-primed allogeneic bone marrow: significantly less graft-versus-host disease and comparable engraftment to G-CSF-mobilized peripheral blood stem cells. Blood 98:3186.

1. A method for inducing transplantation tolerance including the step of administering a G-CSF derivative, or biologically active fragment, homolog or variant thereof, to a donor cell to be transplanted to a recipient.

2. The method of claim 1 wherein the G-CSF derivative, or biologically active fragment, homolog or variant thereof, comprises recombinant G-CSF.

3. The method of claim 2 wherein the recombinant G-CSF comprises recombinant human G-CSF.

4. The method of claim 3 wherein the recombinant human G-CSF comprises recombinant methionyl human G-CSF.

5. The method of claim 4 wherein the recombinant methionyl human G-CSF is non-glycosylated.

6. The method of claim 1 wherein the G-CSF derivative, or biologically active fragment, homolog or variant thereof, comprises peg-G-CSF, or biologically active fragment, homolog or variant thereof.

7. The method of claim 6 wherein the G-CSF derivative, or biologically active fragment, homolog or variant thereof, comprises an N-terminal methionyl residue to which a monomethoxypolyethylene glycol is covalently bound thereto.

8. The method of claim 1 wherein the G-CSF derivative comprises G-CSF or a biologically active G-CSF fragment having a same amino acid sequence as an amino acid sequence of endogenous G-CSF of the donor.

9. The method of claim 1 wherein the G-CSF derivative or biologically active fragment, homolog or variant thereof, is administered to the donor cell in vivo by administering said G-CSF derivative to a donor.

10. The method of claim 9 wherein the G-CSF derivative is administered to the donor as a single dose.

11. The method of claim 9 wherein the G-CSF derivative or biologically active fragment, homolog or variant thereof is administered to the donor in a range from 60 μg/Kg weight of the donor-300 μg/kg weight of the donor.

12. The method of claim 9 wherein the donor is administered between 6 mg-18 mg of the G-CSF derivative or biologically active fragment, homolog or variant thereof, wherein said donor is human.

13. The method of claim 12 wherein the donor is administered 6 mg of the G-CSF derivative or biologically active fragment, homolog or variant thereof.

14. The method of claim 9 wherein the donor cell is isolated from the donor after in vivo administration of the G-CSF derivative or biologically active fragment, homolog or variant thereof.

15. The method of claim 1 wherein the donor cell comprises a cell obtained from an organ, blood or tissue, a single cell suspension, unseparated cells, enriched cells and homogeneous cells.

16. The method of claim 15 wherein the donor cell comprises an immune cell.

17. The method of claim 16 wherein the immune cell is a T cell.

18. The method of claim 17 wherein administering the G-CSF derivative or biologically active fragment, homolog or variant thereof, stimulates the T cell to produce IL-10.

19. The method of claim 18 wherein the T cell is MHC class II restricted.

20. The method of claim 16 wherein the immune cell is a granulocyte-monocyte.

21. The method of claim 20 wherein the granulocyte-monocyte is characterized by a CD11c negative phenotype.

22. The method of claim 21 wherein the granulocyte-monocyte is further characterized by a CD11b^{hi}Gr-1^{dim} phenotype.

23. The method of claim 22 wherein the donor granulocyte-monocyte is further characterized by a MHC Class I positive, MHC Class II positive, CD80 positive, CD86 positive and CD40 negative phenotype.

24. The method of claim 23 wherein the granulocyte-monocyte is capable of stimulating a T cell to produce IL-10.

25. The method of claim 24 wherein the T cell is a donor T cell.

26. The method of claim 15 wherein the donor cell comprises a stem cell.

27. The method of claim 26 wherein the stem cell is obtained from a tissue selected from the group consisting of spleen, blood, bone marrow, skin, nasal tissue and hair follicle.

28. The method of claim 27 wherein the stem cell comprises a hematopoetic stem cell.

29. The method of claim 14 wherein the donor cell is isolated and purified as an enriched cell population.

30. The method of claim 29 wherein the enriched donor cell population comprises a homogeneous cell population.

31. The method of claim 1 wherein the donor cell is isolated from a donor before administering the G-CSF derivative or biologically active fragment, homolog or variant thereof, to the isolated donor cell.

32. The method of claim 29 further including the step of propagating the isolated donor cell in vitro before transplantation of the donor cell to the recipient.

33. The method of claim 1 wherein the donor cell is obtained from a mammal.

34. The method of claim 1 wherein the recipient is a mammal.

35. The method of claim 33 wherein the mammal is a human.

36. The method of claim 1 wherein transplantation tolerance comprises prevention or reduction of graft versus host disease in the recipient.

37. The method of claim 36 wherein the prevention or reduction of graft versus host disease is greater than that provided by administering G-CSF to the donor.

38. A method for stimulating a donor T cell to produce IL-10 including the step of administering a G-CSF derivative or biologically active fragment, homolog or variant thereof, to the donor T cell and a donor granulocyte-monocyte to be transplanted to a recipient.

39. The method of claim 38 wherein the G-CSF derivative or biologically active fragment, homolog or variant thereof comprises recombinant G-CSF.

40. The method of claim 39 wherein the recombinant G-CSF comprises recombinant human G-CSF.

41. The method of claim 40 wherein the recombinant human G-CSF comprises recombinant methionyl human G-CSF.

42. The method of claim 41 wherein the methionyl human G-CSF is not glycosylated.

43. The method of claim 39 wherein the G-CSF derivative or biologically active fragment, homolog or variant thereof, comprises polyethylene glycol.

44. The method of claim 43 wherein the G-CSF derivative, or biologically active fragment, homolog or variant thereof, comprises an N-terminal methionyl residue to which a monomethoxypolyethylene glycol is covalently bound thereto.

45. The method of claim 38 wherein the donor granulocyte-monocyte is characterized by a CD11c negative and a CD11b^{hi}Gr-1^{dim} phenotype.

46. The method of claim 38 wherein the donor T cell and donor granulocyte-monocyte are obtained from a mammal.

47. The method of claim 46 wherein the recipient is a mammal.

48. The method of claim 46 wherein the mammal is a human.

49. The method of claim 38 wherein the G-CSF derivative or biologically active fragment, homolog or variant thereof, is administered in vivo to a donor before transplantation of the donor T cell to the recipient.

50. The method of claim 38 wherein donor non-immune cells in addition to the donor T cells and donor granulocyte-monocyte are transplanted to the recipient.

51. The method of claim 50 wherein donor non-immune cells comprise stem cells.

52. A pharmaceutical composition for inducing immunological tolerance when administered to a subject comprising a G-CSF derivative or biologically active fragment, homolog or variant thereof and a pharmaceutically-acceptable carrier.

53. The pharmaceutical composition of claim 52 wherein the G-CSF derivative or biologically active fragment, homolog or variant thereof comprises recombinant G-CSF.

54. The pharmaceutical composition of claim 53 wherein the recombinant G-CSF comprises recombinant human G-CSF.

55. The pharmaceutical composition of claim 54 wherein the recombinant human G-CSF comprises recombinant methionyl human G-CSF.

56. The pharmaceutical composition of claim 56 wherein the recombinant methionyl human G-CSF is not glycosylated.

57. The pharmaceutical composition of claim 52 wherein the G-CSF derivative comprises peg-G-CSF.

58. The pharmaceutical composition of claim 57 wherein the G-CSF derivative, or biologically active fragment, homolog or variant thereof, comprises an N-terminal methionyl residue to which a monomethoxypolyethylene glycol is covalently bound thereto.

59. The pharmaceutical composition of claim 52 wherein immunological tolerance comprises transplantation tolerance and self-tolerance.

60. The pharmaceutical composition of claim 52 wherein administering the pharmaceutical composition induces greater immunological tolerance when compared with administering G-CSF.

61. The pharmaceutical composition of claim 52 wherein said subject is human.

62. A pharmaceutical composition for inducing immunological tolerance in a subject comprising one or more isolated cells having been administered a G-CSF derivative or biologically active fragment, homolog or variant thereof.

63. The pharmaceutical composition of claim 61 wherein the isolated cell comprises an immune cell.

64. The pharmaceutical composition of claim 62 wherein the immune cell comprises a T cell.

65. The pharmaceutical composition of claim 64 wherein the T cell produces IL-10.

66. The pharmaceutical composition of claim 65 wherein the immune cell comprises a granulocyte-monocyte.

67. The pharmaceutical composition claim 66 wherein the granulocyte-monocyte is characterized by a CD11c negative phenotype.

68. The pharmaceutical composition of claim 67 wherein the granulocyte-monocyte is further characterized by a CD11b^{hi}Gr-1^{dim} phenotype.

69. The pharmaceutical composition of claim 62 wherein said subject is human.

70. The pharmaceutical composition of claim 62 wherein immunological tolerance prevents or reduces graft versus host disease.

71. Use of the pharmaceutical composition of claim 52 to induce immunological tolerance in a patient.

72. A method of transplantation including the steps of:
(1) administering to a donor a pharmaceutical composition comprising a G-CSF derivative or biologically active fragment, homolog or variant thereof and a pharmaceutically-acceptable carrier;
(2) isolating a cell, tissue or organ from said donor; and
(3) transplanting said cell, tissue or organ to a recipient.

73. The method of claim 72 wherein the G-CSF derivative or biologically active fragment, homolog or variant thereof comprise recombinant G-CSF derivative or biologically active fragment, homolog or variant thereof.

74. The method of claim 73 wherein the recombinant G-CSF derivative or biologically active fragment, homolog or variant thereof comprise human G-CSF derivative or biologically active fragment, homolog or variant thereof.

75. The method of claim 72 wherein the G-CSF derivative or biologically active fragment, homolog or variant thereof comprises peg-G-CSF derivative or biologically active fragment, homolog or variant thereof.

76. The method of claim 75 wherein the donor and recipient are human.

77. The method of claim 72 including the steps of isolating cells from the donor and propagating the isolated cells in vitro before transplanting said cells to the recipient.

78. The method of claim 72 wherein transplantation comprises heterologous transplantation whereby the donor and recipient are different individuals.

79. The method of claim 72 wherein transplantation comprises autologous transplantation whereby the donor and recipient are the same individual.

80. A method for inducing self-tolerance in a patient including the step of administering a G-CSF derivative or biologically active fragment, homolog or variant thereof, to the patient.

81. The method of claim 80 wherein inducing self-tolerance in the patient prevents, treats or reduces an autoimmune disorder of the patient.

82. The method of claim 80 wherein the patient is asymptomatic of an autoimmune disorder.

83. The method of claim 81 wherein the autoimmune disorder is selected from the group consisting of rheumatoid arthritis, systemic lupus erythematosus, multiple sclerosis and inflammatory bowel disease.

84. The method of claim 80 wherein the G-CSF derivative or biologically active fragment, homolog or variant thereof stimulates an immune cell of the patient to thereby induce self-tolerance.

85. The method of claim 84 wherein the immune cell comprises a T cell.

86. The method of claim 85 wherein said T cell is stimulated to produce IL-10.

87. The method of claim 84 wherein the immune cell comprises a granulocyte-monocyte cell.

88. The method of claim 87 wherein said granulocyte-monocyte is characterized by a CD11 negative and CD11b^{hi}Gr-1^{dim} phenotype

89. The method of claim 84 wherein the immune cell of the patient is isolated from the patient, propagated in vitro and administered to the patient.

90. The method claim 80 wherein the G-CSF derivative or biologically active fragment, homolog or variant thereof comprises peg-G-CSF or biologically active fragment, homolog or variant thereof.

91. The method of claim 90 wherein the peg-G-CSF comprises peg-human G-CSF or biologically active fragment, homolog or variant thereof.

92. The method of claim 91 wherein the peg-human G-CSF or biologically active fragment, homolog or variant thereof comprises peg-recombinant human G-CSF or biologically active fragment, homolog or variant thereof.
